(19) United States
(12) Patent Application Publication
(10) Pub. No.: US 2023/0270412 A1
(43) Pub. Date: Aug. 31, 2023

(54) METHODS AND DEVICES FOR PREPARATION OF ULTRASOUND CONTRAST AGENTS

(71) Applicant: Lantheus Medical Imaging, Inc., North Billerica, MA (US)

(72) Inventor: Joel Lazewatsky, Sudbury, MA (US)

(73) Assignee: Lantheus Medical Imaging, Inc., North Billerica, MA (US)

(21) Appl. No.: 18/019,310

(22) PCT Filed: Aug. 3, 2021
(86) PCT No.: PCT/US2021/044394

§ 371 Date: Feb. 2, 2023

Related U.S. Application Data
(60) Provisional application No. 63/061,168, filed on Aug. 4, 2020.

(51) Int. Cl. A61B 8/08; A61K 49/22

(52) U.S. Cl. CPC A61B 8/481; A61K 49/223

(57) ABSTRACT

Provided herein are methods and devices for identifying and/or distinguishing UCA formulations and specifically activating such formulations to produce UCA suitable for in vivo use.

### RELATED APPLICATIONS

This application claims the benefit of the filing date of U.S. Provisional Application No. 63/061,168 filed on Aug. 4, 2020, the entire contents of which are incorporated by reference herein.

### BACKGROUND

Contrast-enhanced ultrasound imaging is a commonly used medical imaging modality. Most if not all ultrasound contrast agents (UCA) are gas-filled microspheres that are useful in enhancing ultrasound signal. One such UCA is activated DEFINITY® comprising perflutren lipid microspheres (i.e., perflutren gas encapsulated in lipid microspheres). DEFINITY® formulation is packaged in a vial comprising lipids in an aqueous suspension with perflutren gas in the headspace. Prior to use, DEFINITY® is activated by shaking the vial vigorously, thereby forming lipid microspheres comprising perflutren gas suspended in an aqueous liquid. Proper activation ensures that the microspheres formed are of the appropriate size and concentration to be both diagnostically effective and safe for the subject. Due to the importance of proper size and concentration, activation should optimally be performed in a manner that minimizes the potential for human error.

### SUMMARY

This disclosure contemplates methods and devices for ensuring that activation-dependent UCA formulations, such as but not limited to DEFINITY® and DEFINITY-II formulations, are properly identified and properly activated. As will be appreciated, as additional activation-dependent UCA formulations come to market, it will be imperative to ensure that each is handled and activated in the correct prescribed manner. For example, activation dependent UCA formulations may have different activation speeds, and the device(s) for activating such formulations need to be able to consistently operate at different speeds and for prescribed time periods. In an illustrative example, each activation-dependent UCA may have its own unique activation parameters, including, for example, an activation time and/or activation rate (e.g., shaking rate), and thus it will be imperative that each UCA formulation be handled in a specific manner. Applying incorrect or inconsistent activation parameters to a UCA formulation can result in a UCA that is not diagnostically useful (e.g., due to a very low concentration or inappropriate size of microspheres), requiring a subject to undergo the ultrasound procedure again. At worst, it can result in microspheres that are too large, and this increases the chance of causing ischemia by occluding capillary beds.

According to some embodiments, an apparatus for forming gas-filled microspheres includes a holder arranged to hold a container comprising an ultrasound contrast agent (UCA) formulation, and a shaking means arranged to shake the holder at at least one speed. The shaking means includes a motor arranged to drive movement of the holder via a transmission, wherein at least a portion of the transmission is arranged to prevent slippage during an activation of the UCA formulation. In some embodiments, the transmission is arranged to create friction and/or resistance to movement.

In some embodiments, the transmission includes at least one of a tooth belt, a drive chain, a O-ring, and a gear box.

In some embodiments, the transmission includes a gear having first and second toothed wheels, the first toothed wheel being attachable to the holder and the second toothed wheel being attachable to the motor. In such embodiments, a tooth belt is arranged to engage with each of the first and second toothed wheels. The first toothed wheel may be positioned around a portion of a spindle attachable to the holder, and the second toothed wheel may be positioned around a portion of the motor, such as a shaft. The first and second toothed wheels may be substantially parallel to one another. The first and second toothed wheels also may be substantially co-planar with one another.

In some embodiments, the transmission also includes first and second O-rings, wherein the first O-ring is positioned on a first lateral side of the first toothed wheel and the second O-ring is positioned on a second lateral side of the first toothed wheel. In some embodiments, each of the first and second O-rings and the first toothed wheel are positioned around the spindle.

In some embodiments, the transmission includes first and second profiled wheels, such as sprockets, with teeth or cogs. As with the above, the first wheel may be attachable to the holder and the second wheel may be attachable to the motor. In some embodiments, the first and second wheels engage with a chain, track, or another material that is perforated or indented. For example, the teeth are engageable with the chain, track, or other material with the perforations or indentations.

In some embodiments, the apparatus further includes a housing, the shaking means being disposed in the housing. The apparatus also may include a first identification means arranged to identify the UCA formulation in the vial. The first identification means may include at least one of an RFID reader, a microchip reader, and a barcode scanner. The first identification means also may include at least one antenna.

In some embodiments, the holder may include a shaker arm. The motor may include a direct current (DC) motor, such as a brushless DC motor.

In some embodiments, the at least one speed is pre-determined. The at least one speed may be pre-set by the apparatus. The at least one speed may be less than about 4830 rpm. The at least one speed may be greater than about 4830 rpm. The at least one speed may be about 4530 rpm. The at least one speed may be about 4950 rpm. The at least one speed may include a first speed of about 4530 rpm and a second speed of about 4950 rpm.

In some embodiments, the apparatus is arranged to shake the holder at two or more speeds, wherein the two or more speeds are between about 4300 rpm and about 6000 rpm or optionally between about 4300 rpm and about 5000 rpm.

In some embodiments, the identification means is further arranged to identify a shaking speed for the UCA formulation in the container. The identification means may be arranged to identify the UCA formulation and/or the shaking speed for the UCA formulation by reading an indicator on the container.

According to another embodiment, a shaking device for forming gas-filled microspheres includes a holder arranged to hold a container comprising an ultrasound contrast agent (UCA) formulation, an identification means arranged to identify the UCA formulation in the container; and a shaking means arranged to shake the holder at least one speed, wherein the shaking means includes a motor arranged to drive movement of the holder via a transmission, wherein the transmission includes at least one of a toothed, perforated, indented belt or track, a drive chain, and a gear box.

In some embodiments, the transmission includes a gear having first and second toothed wheels, the first toothed wheel being attachable to the holder and the second toothed wheel being attachable to the motor. In some embodiments, the toothed, perforated, indented belt or track is arranged to engage with each of the first and second toothed wheels.

In some embodiments, the transmission includes first and second profiled wheels, such as sprockets, that are attachable to the motor and holder, respectively. In some embodiments, the toothed, perforated, indented belt or track or other material (e.g., a drive chain) arranged to engage with each of the first and second wheels.

In some embodiments, the device includes a housing, the shaking means being disposed in the housing. In some embodiments, the device includes a first identification means arranged to identify the UCA formulation in the vial. The identification means may include at least one of an RFID reader, a microchip reader, and a barcode scanner. The identification means also may include one or more antennas. The first identification means also may be arranged to identify a shaking speed for the UCA formulation in the container. In some embodiments, the identification means identifies the UCA formulation and/or the shaking speed for the UCA formulation by reading an indicator on the container.

In some embodiments, the holder includes a shaker arm. In some embodiments, the motor includes a DC motor, such as a brushless DC motor.

In some embodiments, the at least one speed is pre-determined. The at least one speed may be pre-set by the shaking device. The at least one speed may be less than 4830 rpm. The at least one speed may be greater than 4830 rpm. The at least one speed may be 4530 rpm. In some embodiments, the at least one speed may be 4950 rpm. In some embodiments, the at least one speed includes a first speed of 4530 rpm and a second speed of 4950 rpm.

In some embodiments, the shaking device is arranged to shake the holder at two or more speeds, wherein the two or more speeds are between about 4300 rpm and about 6000 rpm or optionally between about 4300 rpm and about 5000 rpm.

In some embodiments, a method of using gas-filled lipid microspheres for ultrasound contrast imaging includes activating an ultrasound contrast agent (UCA) formulation to form gas-filled lipid microspheres using a shaking device. The shaking device includes a shaking means with a motor arranged to drive movement of a holder via a transmission, wherein the holder is arranged to shake a container with the UCA formulation at at least one shaking speed, wherein at least a portion of the transmission is arranged to prevent slippage during activation of the UCA formulation. In some embodiments, the transmission is arranged to create friction and/or resistance to movement. The method further includes administering the gas-filled lipid microspheres to a subject, and obtaining an ultrasound contrast image of the subject.

The method may include identifying the at least one shaking speed for the UCA formulation in the container, optionally using the shaking device. The at least one shaking speed may be pre-determined. The at least one shaking speed may be pre-set by the shaking device. The at least one shaking speed may be less than 4830 rpm. The at least one shaking speed may be greater than 4830 rpm. The at least one shaking speed may be 4530 rpm. The at least one shaking speed may be 4950 rpm. In some embodiments, at least one shaking speeds includes a first speed of 4530 rpm and a second speed of 4950 rpm.

In some embodiments, the method includes identifying two or more shaking speeds for the UCA formulation in the container, optionally using the shaking device, wherein the two or more speeds are between about 4300 rpm and about 6000 rpm or optionally between about 4300 rpm and about 5000 rpm.

In some embodiments, activating includes shaking the container via the holder. In some embodiments, the holder includes a shaker arm.

According to another embodiment, a method of using gas-filled lipid microspheres to ultrasound contrast image a subject includes identifying an ultrasound contrast agent (UCA) formulation, activating the UCA formulation to form gas-filled lipid microspheres using a shaking device having a holder arranged to hold a container comprising the UCA formulation, and a shaking means arranged to drive movement of the holder via a motor and a transmission. The transmission includes at least one of a toothed, perforated, indented belt or track, a drive chain, and a gearbox, administering the gas-filled lipid microspheres into a subject, and obtaining an ultrasound contrast image of the subject.

In some embodiments, the container includes an indicator on its external surface. In some embodiments, identifying may include reading the indicator on the external surface of the container via an identification means that is optionally comprised in shaking device. In some embodiments, the identification means includes at least one of an RFID reader, a microchip reader, and a barcode scanner. In some embodiments, the identification means includes at least one antenna.

In some embodiments, the method includes identifying a shaking speed for the UCA formulation in the vial via the identification means, optionally using the identification means. In some embodiments, activating includes shaking the vial via the holder. In some embodiments, the holder includes a shaker arm.

According to another embodiment, a method of using gas-filled lipid microspheres for ultrasound contrast imaging includes activating an ultrasound contrast agent (UCA) formulation to form gas-filled lipid microspheres using an apparatus or shaking device disclosed herein, administering the gas-filled lipid microspheres to a subject, and obtaining an ultrasound contrast image of the subject.

According to still another embodiment, a method of using gas-filled lipid microspheres for ultrasound contrast imaging includes activating an ultrasound contrast agent (UCA) formulation to form gas-filled lipid microspheres using a shaking device disclosed herein, administering the gas-filled lipid microspheres to a subject, and obtaining an ultrasound contrast image of the subject. In some embodiments, the UCA formulation comprises DPPA, DPPC and MPEG5000-DPPE.

According to yet another embodiment, the vial further includes perfluoropropane gas, and the gas-filled microspheres are perfluoropropane gas filled microspheres.

This disclosure provides, in another aspect, a non-transitory computer readable medium programmed with a plurality of instructions that, when executed by at least one processor of a shaking device perform a method, the method comprising: determining based, at least in part, on an identification of a sample type in a vial comprising a UCA formulation inserted into a holder of the shaking device at least one action to perform; and instructing the shaking device to perform the determined at least one action based, at least in part, on the identification.

This disclosure provides, in another aspect, a shaking device comprising: a holder configured to identify a type of sample in a vial comprising a UCA formulation inserted into the holder; at least one storage device configured to store at least one data structure identifying one or more actions to perform for each of a plurality of sample types; at least one processor programmed to access the at least one data structure to determine the one or more actions to perform on the vial based on the identified sample type; and at least one component configured to perform the one or more actions determined by the at least one processor. These and other aspects and embodiments of the invention will be described in greater detail herein.

### BRIEF DESCRIPTION OF THE DRAWINGS

Various embodiments will now be described, by way of example, with reference to the accompanying drawings, in which:

FIG. 1 is a schematic of a sample shaking device and sample container according to embodiments of the present disclosure;

FIG. 2 is a schematic of one embodiment of a sample shaking device;

FIG. 3 is a shaking arm of a sample shaking device according to some embodiments;

FIG. 4 shows a shaking means of a shaking device according to some embodiments;

FIG. 5 is a top view of the shaking means of FIG. 4;

FIG. 6 is a schematic front view of the shaking means of FIG. 4;

FIG. 7 is a perspective view of a portion of a shaking means of a shaking device according to other embodiments;

FIG. 8 is a front view of a portion of a shaking means of a shaking device according to other embodiments;

FIG. 9 is a flowchart of a process for determining action(s) to perform on a sample vial based on its identification according to one aspect;

FIG. 10 is an example of the process of FIG. 9 in which the vial is identified based on an RFID tag associated with the vial;

FIG. 11 is a schematic diagram of a computer system that may be included as a portion of a shaking device for processing a sample container according to one aspect; and

FIG. 12 illustrates measured current versus speed data for a sample shaking device according to some embodiments demonstrating an approximately constant rate of increase of current with increasing motor speed, without the excursions in the current that are indicative of slippage.

### DETAILED DESCRIPTION

Provided herein are methods and activation means (e.g., shaking devices) for facilitating proper and accurate preparation of an activation-dependent UCA formulation. One such UCA formulation is a non-aqueous UCA formulation comprising lipids and a perfluorocarbon gas in a non-aqueous solution. Another such UCA formulation is an aqueous UCA formulation comprising lipids and a perfluorocarbon gas in an aqueous solution. Each of these UCA formulations may provide specific advantages over other, existing UCA formulations including, for example, stability at elevated temperatures (e.g., room temperature) or enhanced safety profiles.

Some UCA formulations have specific activation requirements, some of which are markedly different from the activation requirements of other formulations. For example, it has been found that some UCA formulations described herein must be activated for longer periods of time, at higher speeds, or at varied speeds as compared to other UCA formulations. Typically, devices for activating UCA formulations are operable only at certain speeds and, in certain instances, the speeds remain constant throughout an activation sequence. This may present a challenge in instances where more than one formulation is to be activated using the same device, as the device may need to activate each UCA formulation for its specific required time and for its specific required rate(s). This also may present a challenge in instances where the device is used to activate a UCA formulation at higher speeds or at variable speeds. To that end, the inventors have recognized the need for an activation means cable of consistently activating UCA formulations over a broad range of speeds.

The inventors have discovered that some activation means may experience slippage when the activation means reaches certain shaking speeds and/or varies the shaking speeds during an activation sequence. For example, in some devices, slippage may occur when shaking speeds are above about 4500 rpm, such as above 4530 rpm. The inventors have further discovered that such slippage may affect the ability of the shaking device to consistently activate UCA formulations.

In some embodiments, shaking devices may include a shaking means with a motor arranged to drive movement of a shaker arm holding UCA formulations. In some known embodiments, the shaking means may include a pully and single belt to operably connect the motor and the shaker arm. In such embodiments, the belt may experience slippage when the shaking means reaches certain speeds or varies speeds. For example, in some instances, because different, and possibly higher, speeds are being used by the means, a force applied to the pully and belt may vary during shaking, which may cause slippage of the belt. A radical change in speed also may cause slippage of the belt. As will be appreciated, slippage of the belt may cause movement of the belt relative to the pulleys and/or slippage of one or more pulleys relative to one another. In these embodiments, slippage may result in inconsistent shaking speeds and/or inconsistent preparation of a UCA formulation.

In other embodiments, the inventors have discovered that the activation means may experience resonance when the activation means is at certain speeds and/or varies speeds. For example, existing means may experience heavy load of current consumption. In some embodiments, such resonance may cause the motor to labor at certain speeds, and/or be unable to maintain consistent activation of the UCA formulation.

In view of the above, certain improved devices may include an activation means arranged to consistently withstand shaking at a variety of different speeds, such as, for example, between 4000 rpm and 6000 rpm, and for a variety of times. In some embodiments, the activation means may include a shaking means with one or more features arranged to minimize slippage and/or resist movement as a motor drives movement of a shaker arm holding a UCA formulation. In some embodiments, the shaking means may include a transmission with three or more features arranged to create friction and/or resistance to movement. For example, in some embodiments, the transmission may include a toothed, perforated, indented belt or track, a drive belt, multiple O-rings, a gear box, combinations thereof, or other suitable arrangements. In some embodiments, the toothed, perforated, indented belt or track, the drive belt, the multiple O-rings, and/or the gear box may engage with one or more toothed or profiled wheels. In such embodiments, engagement between the wheels and the toothed, perforated, indented belt or track, the drive belt, the multiple O-rings, and the gear box may minimize movement of the features during shaking. In certain improved devices, the means also may be arranged to minimize resonance.

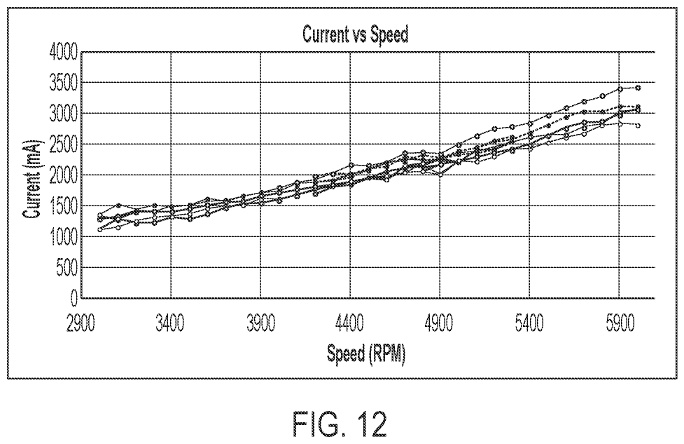

In some embodiments, the means may be able to offer a substantially linear relationship between the motor speed and motor current. In some embodiments, intermittent belt slippage seen when smooth belts, such as those without teeth, or other positive transmission configurations produces large intermittent current excursions in the arm and motor speed at points along the motor speed versus motor current curve. As illustrated in FIG. 12, means described herein, such as those including a positive transmission such as a toothed, perforated, indented belt or track, a drive belt, multiple O-rings, a gear box, combinations thereof, or other suitable arrangements, may eliminate current excursions yielding a relatively smooth increase in motor current with increasing motor speed.

Methods and means disclosed herein may reduce the risk of improper preparation of such UCAs. In some embodiments, UCAs that are not properly and accurately prepared have too few gas-filled microspheres, thereby reducing signal obtainable from such UCAs, at best. At worst, UCAs that are not properly and accurately prepared could cause tissue ischemia by occluding capillary beds, and even patient death. Thus, it is imperative that activation-dependent UCAs are properly handled and prepared. This disclosure provides methods and means including devices that simplify the accurate preparation of activation-dependent UCAs. Unless otherwise stated, the UCA of this disclosure are activation-dependent UCA, and thus the terms “UCA” and “activation-dependent UCA” are used interchangeably.

With the development of UCA formulations that require different activation times, and/or different activation rates (or shaking rates), robust, consistent and error-free product differentiation is required. The methods and means (e.g., devices) provided herein share the unique feature of distinguishing between different activation-dependent UCA formulations. In some embodiments, each activation-dependent UCA formulation may have its own specific activation criteria (or parameters) and therefore each such UCA formulation must be activated in only a certain manner. The methods and means (e.g., devices) provided herein commonly identify and thus distinguish an activation-dependent UCA formulation from other activation-dependent UCA formulations and activate the identified UCA formulation accordingly. This ensures that activation-dependent UCA formulations are activated for a specific period of time and/or at a specific rate (e.g., shaking rate). In some embodiments, the time period and/or rate may be pre-determined, for example, in some instances, specific shaking parameters. In some embodiments, the methods are performed and the means (e.g., devices) operated in relatively autonomous manner such that there is little risk of end user error in the activation process.

An FDA-approved activation-dependent UCA formulation is DEFINITY®. As described in greater detail below, DEFINITY® is provided in a vial as an aqueous suspension of lipids with a perflutren gas headspace. When activated for its prescribed period of time of seconds using a VIALMIX® (or VIALMIX® device, as the terms are used interchangeably), “activated DEFINITY®” comprises a maximum of 1.2×10¹⁰ perflutren lipid microspheres per ml of suspension. Activation for the wrong duration or shake speed will impact the microsphere profile, and render the UCA suboptimal or unusable in some instances. With the advent of at least one additional activation-dependent UCA formulation, in the form of a non-aqueous activation-dependent UCA formulation described below, it is important to ensure there is no confusion between different activation-dependent UCA formulations and that each is handled and activated properly, given the adverse consequences that can otherwise ensue.

As an example, certain improved devices may include a holder for holding a container with a UCA formulation and a means for shaking the holder, the means including a motor arranged to drive movement of the holder via a transmission. In some embodiments, the transmission may be arranged to minimize slippage during activation of the UCA formulation. In some embodiments, as described herein, the transmission may include a gear with a tooth belt. The transmission also may include a pulley with multiple O-rings. The transmission also may include sprockets and a drive chain. In still other embodiments, the transmission may include a gear box.

In some embodiments, certain improved devices may comprise counters that can monitor use of the device, including lifetime use of the device, that can be useful in avoiding mechanical malfunction at critical times. For example, in some embodiments, the device may be arranged to turn off, and remain turned off, when a certain number of activations is performed. Certain improved devices also may include a vibration/isolation damper arranged to protect the shaking means (e.g., motor) during transport. Certain improved devices also may include an interlock arranged to ensure device is ready for activation of the UCA formulation.

In some embodiments, certain improved devices may include one or more alerts, such as if the device is not properly functioning. For example, the device may be arranged to alert a user if the device does not turn on and/or if the shaking speed is too fast or too slow.

As described in greater detail herein, some of these devices may also be able to identify and activate more than one UCA formulation, and may therefore be capable of identifying and optionally distinguishing between two or more UCA formulations. In this latter respect, the device may automatically recognize a container comprising a UCA formulation and based on such identity, which may be imparted for example by the label, shape, color or size of the container, or the optical properties of its contents, may activate the UCA formulation for a pre-determined period of time which in turn may be selected between two or more different pre-determined periods of time. The device may be able to perform such recognition with no or minimal user input.

Certain improved devices also may include a shaker arm arranged to reduce failure when the device reaches certain speeds and/or varies speeds. For example, in some embodiments, the shaker arm geometry may be configured to reduce stress concentrations. In an illustrative embodiment, the arm may have one or more stiffening ribs arranged to reduce stress concentrations. The arm also may be formed of a material with improved thermal stability and strength, such that the arm resists thermal degradation during shaking.

Certain improved devices also may be arranged to minimize, or event eliminate, interference from stray signals (e.g., RFID signals). For example, the device may be formed of or coated with a material that may shield such interferences.

### Activation-Dependent UCAs

As used herein, a UCA refers to gas-filled microspheres that are useful in enhancing ultrasound signal. In most instances, the UCA is provided in solution such as a pharmaceutically acceptable solution. Depending on the concentration of microspheres in the UCA, it may be diluted with a pharmaceutically acceptable carrier prior to administration to a subject, although this may not be required in some instances.

An activation-dependent UCA formulation, as used herein, refers to a composition that must be activated in order to form gas-filled microspheres. A UCA formulation typically contains no such gas-filled microspheres (or such a low concentration of them to not be clinically useful), and must be activated in order to form microspheres of sufficient diameter and concentration to be clinically useful.

Activation-dependent UCA formulations typically require vigorous shaking prior to use to form gas-filled microspheres. Such activation is performed by an end user or an intermediate, but not the supplier or manufacturer of the UCA formulation. Activation-dependent UCA formulations are typically packaged in vials that minimally house a lipid solution and a gas. The shaking of the lipid solution and the gas results in the formation of gas-filled microspheres that act as the contrast agent in an ultrasound imaging procedure.

Unless otherwise stated, the UCA formulations of this disclosure are activation-dependent UCA formulations, and thus the terms “UCA formulation” and “activation-dependent UCA formulation” are used interchangeably.

By “gas-filled”, as used herein, it is meant the microspheres comprise gas, such as a perfluorocarbon gas including but not limited to perflutren gas, in their internal cavity. The lipid shell that encapsulates the gas may be arranged as a unilayer or a bilayer, including unilamellar or multilamellar bilayers. The microspheres may have a mean diameter of less than 10 microns, or less than 6 microns, or less than 3 microns, or more preferably less than 2 microns. These mean diameters intend that, when a population of microspheres is analyzed, the mean diameter of the population is less than 10 microns, or less than 6 microns, or less than 3 microns, or more preferably less than 2 microns. The microspheres may have a mean diameter in the range of 0.5 to 3 microns, or 1 to 2 microns, or 1.4 to 1.8 microns, or 1.4 to 1.6 microns. The mean diameter may be about 1.6 microns.

Prior to use, an activation-dependent UCA formulation must be shaken vigorously, to form gas-filled microspheres. In some instances, the microspheres may be combined with, for example, an aqueous solution prior to withdrawal from their container. This is particularly the case with microspheres made from non-aqueous UCA formulations. Such a step is referred to as reconstitution, in the context of this disclosure. In some instances, the microspheres, whether or not reconstituted, may be withdrawn from their container and combined in another solution, such as an aqueous solution, prior to administration to a subject. Such a step is referred to as dilution, in the context of this disclosure. The reconstituted population of microspheres may be used neat or after dilution in a pharmaceutically acceptable solution. Such dilution may be about 10-fold up to and about 50-fold, although it is not so limited.

As used herein, gas-filled microspheres and lipid-encapsulated gas microspheres are used interchangeably.

UCA formulations minimally comprise one or more lipid types and a gas such as perfluorocarbon gas such as perflutren gas. As described in greater detail herein, UCA formulations include aqueous UCA formulations such as DEFINITY® and non-aqueous UCA formulations such as DEFINITY-II. DEFINITY® comprises lipids DPPA, DPPC and MPEG5000-DPPE, propylene glycol and glycerol in an aqueous solution together with perflutren gas. DEFINITY-II, on the other hand, comprises lipids DPPA, DPPC and MPEG5000-DPPE, and propylene glycol and glycerol together with a perfluorocarbon gas (e.g., perflutren gas).

### DEFINITY®

DEFINITY® is an example of an aqueous UCA formulation. Activated DEFINITY® is approved by the FDA for use in subjects with suboptimal echocardiograms to opacify the left ventricular chamber and to improve the delineation of the left ventricular endocardial border. DEFINITY® is provided in a vial comprising a single phase solution comprising DPPA, DPPC and MPEG5000-DPPE in a 10:82:8 mole % ratio in an aqueous solution, and a headspace comprising perfluoropropane gas. Prior to its administration to a subject, DEFINITY® is activated by vigorous shaking, such as vigorous mechanical shaking, and is thereafter referred to as “activated DEFINITY®”. Activation results in the formation of a sufficient number of lipid-encapsulated gas microspheres having an average diameter of 1.1 to 3.3 microns. DEFINITY® however must be refrigerated until just prior to use. This limits its utility particularly in settings that lack appropriate refrigeration, particularly during the storage period.

In other aqueous UCA formulations, DPPA, DPPC and DPPE may be used in molar percentages of about 77-90 mole % DPPC, about 5-15 mole % DPPA, and about 5-15 mole % DPPE, including DPPE-MPEG5000. Preferred ratios of each lipid include weight % ratios of 6.0 to 53.5 to 40.5 (DPPA:DPPC:MPEG5000-DPPE) or a mole % ratio of 10 to 82 to 8 (10:82:8) (DPPA:DPPC:MPEG5000-DPPE).

In still other aqueous UCA formulations, DPPA may be absent. As an example, other aqueous UCA formulations may comprise DPPC and DPPE, and optionally MPEG5000-DPPE. These may be used in molar percentages of about 77-90 mole % DPPC, about 5-15 mole % DPPE, and about 5-15 mole % MPEG5000-DPPE. Examples of ratios of each lipid include weight % ratios of 6.0 to 53.5 to 40.5 (DPPE:DPPC:MPEG5000-DPPE) or a mole % ratio of 10 to 82 to 8 (10:82:8) (DPPE:DPPC:MPEG5000-DPPE).

DEFINITY-II and other non-aqueous UCA formulations Contemplated herein are a variety of non-aqueous UCA formulations. Some such formulations comprise a non-aqueous mixture of one or more lipids and propylene glycol (PG), or glycerol (G), or propylene glycol and glycerol (PG/G). These formulations may be stored at higher temperatures (e.g., room temperature) for longer periods of time than were previously thought possible, without significant degradation. The non-aqueous UCA formulations, for example DEFINITY-II, may comprise less than 10%, less than 5%, or less than 2% impurities when stored at room temperature for a period of time, including for example, about 1 month, about 2 months, about 3 months, about 6 months, or longer including about 1 year, or about 2 years. Significantly, the non-aqueous UCA formulations may comprise fewer impurities than DEFINITY® when both formulations are stored at room temperature (i.e., when the non-aqueous UCA formulation and DEFINITY® formulation are stored at room temperature). This reduction in impurity level may be a difference of about 1%, about 2%, about 3%, about 4%, or about 5%, or more.

The non-aqueous mixture of lipids in propylene glycol, or glycerol, or propylene glycol and glycerol may be a mixture having less than or equal to 5% water by weight (i.e., weight of water to the weight of the combination of lipids and propylene glycol and/or glycerol). In some instances, the non-aqueous mixture comprises less than 5% water (w/w), 1-4% water (w/w), 1-3% water (w/w), 2-3% water (w/w), or 1-2% water (w/w). In some instances, the non-aqueous mixture comprises less than 1% water (w/w). The water content may be measured at the end of manufacture (and prior to long term storage) or it may be measured after storage, including long term storage, and just before use.

The non-aqueous mixture also may be salt-free intending that it does not contain any salts other than lipid counter-ions. More specifically, and as an example, lipids such as DPPA and DPPE are typically provided as sodium salts. As used herein, a salt-free non-aqueous mixture may comprise such counter-ions (e.g., sodium if DPPA and/or DPPE are used) but they do not contain other ions. In some instances, the non-aqueous mixture is free of sodium chloride or chloride.

The non-aqueous mixture may comprise a buffer. The buffer may be an acetate buffer, a benzoate buffer, or a salicylate buffer, although it is not so limited. Non-phosphate buffers are preferred in some instances due to their dissolution profiles in the non-aqueous mixtures provided herein. In some instances, a phosphate buffer may be used (e.g., following or concurrent with addition of aqueous diluent such as the reconstitution or dilution step, as discussed earlier).

In some embodiments, the non-aqueous mixture comprises, consists of, or consists essentially of (a) one or more lipids, (b) propylene glycol, or glycerol, or propylene glycol/glycerol, and (c) a non-phosphate buffer. Such non-aqueous mixtures may be provided together with a gas such as a perfluorocarbon gas or they may be provided alone (i.e., in the absence of a gas). Such non-aqueous mixtures may be provided in single use amounts and/or in single use containers, with or without a gas. Such containers will typically be sterile.

The non-phosphate buffer may be, but is not limited to, an acetate buffer, a benzoate buffer, a salicylate buffer, a diethanolamine buffer, a triethanolamine buffer, a borate buffer, a carbonate buffer, a glutamate buffer, a succinate buffer, a malate buffer, a tartrate buffer, a glutarate buffer, an aconite buffer, a citrate buffer, a lactate buffer, a glycerate buffer, a gluconate buffer, and a tris buffer. It is within the skill of the ordinary artisan to determine and optimize the concentration of buffer for each buffer type.

If DPPA, DPPC and DPPE are used, they may be used in molar percentages of about 77-90 mole % DPPC, about 5-15 mole % DPPA, and about 5-15 mole % DPPE, including DPPE-PEG5000. Preferred ratios of each lipid include weight % ratios of 6.0 to 53.5 to 40.5 (DPPA:DPPC:MPEG5000-DPPE) or a mole % ratio of 10 to 82 to 8 (10:82:8) (DPPA:DPPC:MPEG5000-DPPE).

If DPPC and DPPE, and optionally MPEG5000-DPPE are used, they may be used in molar percentages of about 77-90 mole % DPPC, about 5-15 mole % DPPE, and about 5-15 mole % MPEG5000-DPPE. Examples of ratios of each lipid include weight % ratios of 6.0 to 53.5 to 40.5 (DPPE:DPPC:MPEG5000-DPPE) or a mole % ratio of 10 to 82 to 8 (10:82:8) (DPPE:DPPC:MPEG5000-DPPE). In some instances, the lipid concentration may range from about 0.1 mg to about 20 mg per mL of non-aqueous mixture, including about 0.9 mg to about 10 mg per mL of non-aqueous mixture and about 0.9 mg to about 7.5 mg per mL of non-aqueous mixture. In some embodiments, the lipid concentration may range from about 0.94 mg to about 7.5 mg lipid per mL of non-aqueous mixture, including about 1.875 mg to about 7.5 mg lipid per mL of non-aqueous mixture, or about 3.75 mg to about 7.5 mg lipid per mL of non-aqueous mixture. In some instances, the lipid concentration is about 0.94 mg to about 1.875 mg per mL of non-aqueous mixture, about 1.875 mg to about 3.75 mg per mL of non-aqueous mixture, or about 3.75 mg to about 7.5 mg of total lipid per mL of non-aqueous mixture.

As an example, the lipid concentration may range from about 0.1 mg to about 10 mg lipid per mL of propylene glycol/glycerol (combined), including about 1 mg to about 5 mg lipid per mL of propylene glycol/glycerol (combined). In some instances, the lipid concentration is about 0.94 mg to about 3.75 mg lipid per mL of propylene glycol/glycerol (combined).

As another example, the lipid concentration may range from about 0.1 mg to about 20 mg lipid per mL of propylene glycol, including about 1 mg to about 10 mg lipid per mL of propylene glycol, or about 2 mg to about 7.5 mg lipid per mL of propylene glycol, or about 3.75 mg to about 7.5 mg lipid per ml of propylene glycol. In some embodiments, the lipid concentration is about 1.875 mg to about 7.5 mg lipid per mL of propylene glycol, including about 3.75 mg to about 7.5 mg lipid per mL of propylene glycol.

As yet another example, the lipid concentration may range from about 0.1 mg to about 20 mg lipid per mL of glycerol, including about 1 mg to about 10 mg lipid per mL glycerol, or about 2 mg to about 7.5 mg lipid per mL of glycerol, or about 3.75 mg to about 7.5 mg lipid per ml of glycerol. In some instances, the lipid concentration is about 1.875 mg to about 7.5 mg lipid per mL of glycerol, including about 3.75 mg to about 7.5 mg lipid per mL of glycerol.

DEFINITY-II comprises lipids DPPA, DPPC and MPEG5000-DPPE at a mole % ratio of 10 to 82 to 8 (10:82:8) and a total lipid content of 3.75 mg/mL, and propylene glycol (517.5 mg/mL), glycerol (631 mg/mL), Sodium acetate (0.370 mg/mL), Acetic acid (0.030 mg/mL) together with a perfluoropropane (perflutren) gas headspace (6.52 mg/mL).

The microspheres may be reconstituted or diluted in an aqueous diluent, and such aqueous diluent may comprise salts such as but not limited to sodium chloride, and thus may be regarded as a saline solution. The aqueous diluent may comprise a buffer such as a phosphate buffer, and thus may be regarded as a buffered aqueous diluent. The aqueous diluent may be a buffered saline solution. The non-aqueous mixture may comprise a buffer such as a non-phosphate buffer, examples of which are provided herein. The non-aqueous mixture and the aqueous diluent may both comprise a buffer. In typical embodiments, either the non-aqueous mixture or the aqueous diluent comprises a buffer, but not both. The buffer concentration will vary depending on the type of buffer used, as will be understood and within the skill of the ordinary artisan to determine. The buffer concentration in the non-aqueous lipid formulation may range from about 1 mM to about 100 mM. In some instances, the buffer concentration may be about 1 mM to about 50 mM, or about 1 mM to about 20 mM, or about 1 mM to about 10 mM, or about 1 mM to about 5 mM, including about 5 mM.

The final formulation to be administered, typically intravenously, to a subject including a human subject may have a pH in the range of 4-8 or in a range of 4.5-7.5. In some instances, the pH may be in a range of about 6 to about 7.5, or in a range of 6.2 to about 6.8. In still other instances, the pH may be about 6.5 (e.g., 6.5+/−0.5 or +/−0.3). In some instances, the pH may be in a range of 5 to 6.5 or in a range of 5.2 to 6.3 or in a range of 5.5 to 6.1 or in a range of 5.6 to 6 or in a range of 5.65 to 5.95. In still another instance, the pH may be in a range of about 5.7 to about 5.9 (e.g., +/−0.1 or +/−0.2 or +/−0.3 either or both ends of the range). In another instance, the pH may be about 5.8 (e.g., 5.8+/−0.15 or 5.8+/−0.1).

In some embodiments, the aqueous diluent comprises glycerol, a buffer such as phosphate buffer, salt(s) and water. Such an aqueous diluent may be used with a non-aqueous mixture that lacks glycerol. In some embodiments, the lipid solution further comprises saline (salt(s) and water combined) and glycerol in a weight ratio of 8:1.

In some embodiments, the aqueous diluent comprises propylene glycol, a buffer such as phosphate buffer, salt(s) and water. Such an aqueous diluent may be used with a non-aqueous mixture that lacks propylene glycol.

In some embodiments, the aqueous diluent comprises a buffer such as phosphate buffer, salt(s) and water. Such an aqueous diluent may be used with a non-aqueous mixture that comprises both propylene glycol and glycerol.

The microspheres may be reconstituted and used directly (neat) or they may be reconstituted and diluted. Reconstitution and dilution involve combining the microspheres with an aqueous solution, such as a pharmaceutically acceptable solution. Either step or both together may yield microsphere concentrations of at least 1×10⁷ microspheres per ml of solution, or at least 5×10⁷ microspheres per ml of solution, or at least 7.5×10⁷ microspheres per ml of solution, or at least 1×10⁸ microspheres per ml of solution, or at least 1×10⁹ microspheres per ml of solution, or about 5×10⁹ microspheres per ml of solution. The range of microsphere concentration may be, in some instances, 1×10⁷ to 1×10¹⁰ microspheres per ml of solution, and more typically 5×10⁷ to 5×10⁹ microspheres per ml of solution. A reconstituted population of microspheres may be further diluted about 10-fold up to and about 50-fold, without limitation.

In some instances, activation of the non-aqueous UCA formulation followed by reconstitution yields about 4-5×10⁹ microspheres per ml of solution, which may be diluted about 10 fold to yield about 4-5×10⁸ microspheres per ml of solution.

DEFINITY-II is described in greater detail in PCT Application PCT/US2015/067615, the entire contents of which are incorporated by reference herein.

DEFINITY-II is contemplated for use in a manner identical to that of DEFINITY®. Thus, for example, DEFINITY-II may be used in subjects with suboptimal echocardiograms to opacify the left ventricular chamber and to improve the delineation of the left ventricular endocardial border, among other imaging applications.

### Other Aqueous UCA Formulations

Other aqueous UCA formulations are being developed. Some new aqueous UCA formulations comprise, relative to DEFINITY®, a smaller volume of aqueous lipid solution (i.e., the aqueous solution comprising lipids) and a larger gas headspace. Other new aqueous UCA formulations comprise, relative to DEFINITY®, a lower lipid concentration in the aqueous solution. And still other aqueous UCA formulations are provided in containers of various shape and size (and thus volume), relative to DEFINITY®. All of these new aqueous UCA formulations can be activated to yield gas-filled microspheres on par with activated DEFINITY®, including mean diameter profile, without compromising the acoustic properties of the microspheres. The ability to form lipid-encapsulated gas microspheres suitable for clinical use using substantially less lipid by reducing either the volume of lipid solution or the lipid concentration is beneficial for a number of reasons, including reducing material wastage and the likelihood of overdosing a subject. The choice of container would allow the end user to select the most convenient shape and size (volume) for their desired application.

An example of one such new aqueous UCA formulation, referred to herein as DEFINITY-III, comprises lipids DPPA, DPPC and PEG5000-DPPE (where PEG5000 includes without limitation hydroxy-PEG5000 or MPEG5000) in an aqueous solution together with a perfluorocarbon gas (e.g., perflutren gas) in a container, wherein the perfluorocarbon gas occupies about 60-85% of the container volume. DEFINITY®, in contrast, is provided in a container (i.e., a vial) wherein the perfluorocarbon gas (i.e., perflutren gas) occupies about 54% of the container volume.

Another example of new aqueous UCA formulation, referred to herein as DEFINITY-IV comprises an aqueous lipid solution comprising about 0.1 mg to about 0.6 mg of DPPA, DPPC and PEG5000-DPPE (combined) per ml of solution, and a perfluorocarbon gas, in a container.

Still other formulations are DPPA-less, intending that they lack DPPA. They may include DPPC and DPPE, and optionally further include MPEG5000-DPPE. Exemplary ratios of lipids in some of these formulations are provided herein.

These new aqueous UCA formulations including DEFINITY-III and DEFINITY-IV are described in greater detail in PCT Application PCT/US2014/063267, the entire contents of which are incorporated by reference herein.

### Activation

UCA formulations are vigorously shaken to form gas-filled microspheres which will typically be used as UCA. Such gas-filled microspheres may be formed directly or they may be formed through a process that involves formation of microspheres and incorporation of gas into such microspheres. Activation is typically carried out by vigorously shaking of a container (e.g., a vial) comprising a UCA formulation. The UCA formulation minimally comprises lipids and gas, and thus activation minimally results in gas-filled lipid microspheres. The lipids may be present in an aqueous solution such as is the case with DEFINITY®, DEFINITY-III and DEFINITY-IV or they may be present in a non-aqueous solution such as is the case with novel UCA formulations including for example DEFINITY-II, described in greater detail herein. Thus, in some instances, activation comprises shaking an aqueous solution comprising a lipid in the presence of a gas, such as a perfluorocarbon gas (e.g., perflutren). In other instances, activation comprises shaking a non-aqueous solution comprising a lipid in the presence of a gas, a perfluorocarbon gas (e.g., perflutren). It is to be understood that perflutren, perflutren gas and octafluoropropane are used interchangeably herein.

Shaking, as used herein, refers to a motion that agitates a solution, whether aqueous or non-aqueous, such that gas is introduced from the local ambient environment within the container (e.g., vial) into the solution. Any type of motion that agitates the solution and results in the introduction of gas may be used for the shaking. The shaking must be of sufficient force or rate to allow the formation of foam after a period of time. Preferably, the shaking is of sufficient force or rate such that foam is formed within a short period of time, as prescribed by the particular UCA formulation. Thus in some instances such shaking occurs for about 30 seconds, or for about 45 seconds, or for about 60 seconds, or for about 75 seconds, or for about 90 seconds, or for about 120 seconds, including for example for 30 seconds, or for 45 seconds, or for 60 seconds, or for 75 seconds, or for 90 seconds, or for 120 seconds. In some instances, the activation may occur for a period of time in the range of 60-120 seconds, or in the range of 90-120 seconds.

The disclosure contemplates that, in some instances, the shaking time (or duration) will vary depending on the type of UCA formulation being activated. For example, in some instances, an aqueous UCA formulation may be shaken for shorter periods of time than a non-aqueous UCA formulation. The disclosure also contemplates that, in such instances, the shaking rate (or shaking speed, as those terms are used interchangeably herein) may be constant. Thus, an activation means such as a shaking device may be set to shake at one rate (defined in terms of number of shaking motions per minute, for example) for two or more different pre-determined periods of time.

The disclosure further contemplates that, in some instances, the shaking rate will vary depending on the type of UCA formulation being activated. For example, in some instances, an aqueous UCA formulation may be shaken at a slower shaking rate than a non-aqueous

UCA formulation. The disclosure contemplates that, in such instances, the shaking time (or duration, as those terms are used interchangeably herein) may be constant. Thus, an activation means such as a shaking device may be set to shake at two or more different pre-determined shaking rates (defined in terms of number of shaking motions per minute, for example) for one set period of time.

The disclosure further contemplates that, in some instances, the shaking time and the shaking rate will vary depending on the type of UCA formulation being activated. For example, in some instances, an aqueous UCA formulation may be shaken for a first period of time at a first shaking rate and a non-aqueous UCA formulation may be shaken for a second period of time at a second shaking rate, and the first and second periods of time may be different and the first and second shaking rates may be different. Thus, an activation means such as a shaking device may be set to shake at one or more different pre-determined shaking rates (defined in terms of number of shaking motions per minute, for example) for one or more different pre-determined periods of time. For example, an activation means such as a shaking device may be set to shake at (1) a first pre-determined shaking rate for a first pre-determined period of time and (2) a second pre-determined shaking rate for a second pre-determined period of time, and the first and second periods of time are different and the first and second shaking rates are different. DEFINITY® activation requires vigorous shaking for about 45 seconds with a VIALMIX®. Unless indicated otherwise, the term “about” with respect to activation time intends a time that is +/−20% of the noted time (i.e., 45+/−9 seconds).

DEFINITY-II may be activated with a VIALMIX® for periods of time ranging from 60 to 120 seconds. In some instances, DEFINITY-II is activated for about 75 seconds (i.e., 75+/−15 seconds). DEFINITY-II may be activated for longer periods of time including 90-120 seconds

The shaking may be by swirling (such as by vortexing), side-to-side, or up and down motion. Further, different types of motion may be combined. The shaking may occur by shaking the container (e.g., the vial) holding the aqueous or non-aqueous lipid solution, or by shaking the aqueous or non-aqueous solution within the container (e.g., the vial) without shaking the container (e.g., the vial) itself. Shaking is carried out by machine in order to standardize the process. Mechanical shakers are known in the art and their shaking mechanisms or means may be used in the devices of the present disclosure. Examples include amalgamators such as those used for dental applications. Vigorous shaking encompasses at least 1000, at least 2000, at least 3000, at least 4000, at least 4500, at least 5000, at least 6000 or more shaking motions per minute. In some instances, vigorous shaking includes shaking in the range of 4000-4800 shaking motions per minute. For example, VIALMIX® targets shaking for 4530 “figure of eight” revolutions per minute, and tolerates shaking rates in the range of 4077-4756 revolutions per minute. Other UCA formulations may include shaking in the range of less than 4830, such as 4530. Other shaking speeds may include shaking in the range of 4830 to 6000 rpm. For example, shaking may include a shaking speed of about 4950. Other shaking speeds in the range of 4000-6000 shaking motions per minute may be performed via the shaking means. Vortexing may encompass at least 250, at least 500, at least 750, at least 1000 or more revolutions per minute. Vortexing at a rate of at least 1000 revolutions per minute is an example of vigorous shaking, and is more preferred in some instances. Vortexing at 1800 revolutions per minute is most preferred.

The shaking rate can influence the shaking duration needed. A faster shaking rate will tend to shorten the duration of shaking time needed to achieve optimal microbubble formation. For example, shaking at 4530 rpm for a 45 second duration will achieve 3398 total revolutions on a VIALMIX®. Shaking at 3000 rpm would require 68 seconds to achieve the same number of revolutions. It will also be understood, therefore, that a slower shaking rate will tend to lengthen the duration of shaking time needed to achieve optimal microbubble formation. The duration and shake speed required will also be influenced by the shape of the travel path and amplitude of shaking. The velocity the liquid in the container reaches and the forces exerted upon change of direction will influence gas incorporation. These aspects will be impacted upon based on the shaker arm length and path, the container shape and size, the fill volume and the formulation viscosity. Water has a viscosity of approximately 1.14 cps at 15° C. (Khattab, I. S. et al., Density, viscosity, surface tension, and molar volume of propylene glycol+water mixtures from 293 to 323 K and correlations by the Jouyban-Acree model Arabian Journal of Chemistry (2012). In contrast, propylene glycol has a viscosity of 42 cps at 25° C. (Khattab, I. S. et al., Density, viscosity, surface tension, and molar volume of propylene glycol+water mixtures from 293 to 323 K and correlations by the Jouyban-Acree model Arabian Journal of Chemistry (2012) and glycerol has a viscosity of 2200 cps at 15° C. (Secut J B, Oberstak H E Viscosity of Glycerol and Its Aqueous Solutions. Industrial and Engineering Chemistry 43. 9 2117-2120 1951). DEFINITY-II has a high viscosity of 1150 cps at 15° C. Since DEFINITY® is predominantly water it has a much lower viscosity than DEFINITY-II.

The formation of gas-filled microspheres upon activation can be detected by the presence of a foam on the top of the aqueous or non-aqueous solution and the solution becoming white.

Activation is carried out at a temperature below the gel state to liquid crystalline state phase transition temperature of the lipid employed. By “gel state to liquid crystalline state phase transition temperature”, it is meant the temperature at which a lipid layer (such as a lipid monolayer or bilayer) will convert from a gel state to a liquid crystalline state. This transition is described for example in Chapman et al., J. Biol. Chem. 1974 249, 2512-2521. The gel state to liquid crystalline state phase transition temperatures of various lipids will be readily apparent to those skilled in the art and are described, for example, in Gregoriadis, ed., Liposome Technology, Vol. I, 1-18 (CRC Press, 1984) and Derek Marsh, CRC Handbook of Lipid Bilayers (CRC Press, Boca Raton, Fla. 1990), at p. 139. Vigorous shaking can cause heating of the formulation based on the shake speed, duration, shaker arm length and path, the container shape and size, the fill volume and the formulation viscosity.

It will be understood by one skilled in the art, in view of the present disclosure, that the lipid(s) or lipid microspheres may be manipulated prior to or subsequent to being subjected to the methods provided herein. For example, after the shaking is completed, the gas-filled microspheres may be extracted from their container (e.g., vial). Extraction may be accomplished by inserting a needle of a syringe or a needle-free spike (e.g., PINSYNC®) into the container, including into the foam if appropriate, and drawing a pre-determined amount of liquid into the barrel of the syringe by withdrawing the plunger or by adding an aqueous liquid, mixing and drawing a pre-determined amount of liquid into the barrel of the syringe by withdrawing the plunger. As another example, the gas-filled microspheres may be filtered to obtain microspheres of a substantially uniform size. The filtration assembly may contain more than one filter which may or may not be immediately adjacent to each other.

### Methods

Accordingly, this disclosure provides various methods for forming gas-filled microspheres. In some instances, these methods minimally comprise activating an activation-dependent UCA formulation to form gas-filled microspheres. Activation may be performed using an activation means (e.g., a shaking device). Such activation means may be capable of activation alone or it may be capable of identification of a UCA formulation (or its container) and activation of such formulation. Thus, some methods comprise identifying a UCA formulation and then activating such UCA formulation based on its identity. In some embodiment, a single activation means (e.g., device) may perform both the identification and activation steps. Alternatively, different means may be used to perform each step. In still another embodiment, a means may be used to only activate the formulation.

In some instances, these methods comprise activating an activation-dependent UCA formulation to form gas-filled microspheres using means (e.g., a device) that identifies a non-aqueous UCA formulation. Identification of a non-aqueous UCA formulation may involve reading a label specific to a non-aqueous UCA formulation. The activation means may be set to hold and activate the non-aqueous UCA formulation for a pre-determined period of time. In some embodiments, such pre-determined period of time is about 75 seconds.

In other instances, these methods comprise activating an activation-dependent UCA formulation to form gas-filled microspheres using a means that distinguish a non-aqueous UCA formulation from an aqueous UCA formulation (or alternatively, a means that distinguish an aqueous UCA formulation from a non-aqueous UCA formulation).

An aqueous UCA formulation is an aqueous solution comprising one or more lipid(s) and a gas. Upon activation, the lipids and gas together form the gas-filled microspheres. Examples of an aqueous UCA formulation are DEFINITY®, DEFINITY-III, and DEFINITY-IV.

A non-aqueous UCA formulation is a non-aqueous solution comprising one or more lipid(s) and a gas. Upon activation, the lipids and gas together form the gas-filled microspheres although in this case the microspheres are surrounded by a non-aqueous solution. An example of a non-aqueous UCA formulation is a room temperature stable formulation referred to herein as DEFINITY-II. As described in greater detail herein, DEFINITY-II minimally comprises lipids DPPA, DPPC and PEG5000-DPPE in propylene glycol and glycerol, along with a buffer and octafluoropropane (perflutren) gas. PEG5000 refers to PEG having a molecular weight of 5000 Daltons. It may be hydroxy-PEG or methoxy-PEG. In some embodiments, DEFINITY-II comprises MPEG5000-DPPE Thus examples of non-aqueous UCA formulations comprise, for example, lipids DPPA, DPPC and MPEG5000-DPPE, propylene glycol, glycerol, a buffer, and octafluoropropane (perflutren) gas; or lipids DPPA, DPPC and MPEG5000-DPPE, propylene glycol, a buffer, and octafluoropropane (perflutren) gas; or lipids DPPA, DPPC and MPEG5000-DPPE, glycerol, a buffer, and octafluoropropane (perflutren) gas; or lipids DPPA, DPPC and MPEG5000-DPPE, propylene glycol, glycerol, and octafluoropropane (perflutren) gas. Once activated, the gas-filled microspheres similarly comprise a DPPA/DPPC/MPEG5000 DPPE lipid shell that encapsulates the perflutren gas. These microspheres however are diluted in an aqueous solution, such as an aqueous saline solution and then administered to a subject, either as a bolus or continuous infusion injection.

Significantly, it has been found that these aqueous and non-aqueous UCA formulations have different optimal activation times in order to obtain diagnostically suitable gas-filled microspheres. For example, in some instances the shaking rate is about 4530 shaking motions (e.g., figure of eight motions) per minute and shaking is performed using a VIALMIX®. Some aqueous UCA formulations, including DEFINITY®, are activated in about 45 seconds while some non-aqueous UCA formulations, such as DEFINITY-II, are activated in 60-120 seconds and in some instances in about 75 seconds in order to achieve a substantially similar microsphere profile with respect to size distribution, using the same shaking rate of about 4530 rpm. Alternatively, the DEFINITY-II may be activated at a higher shaking rate and for a shorter period of time, relative to DEFINITY-I or another aqueous formulation. For example, DEFINITY-II may be activated at a shaking rate of about 4800-5100 rpm, or about 4850-5050 rpm, or about 4900-5000 rpm, or about 4950 rpm. The activation time may be about 35-55 seconds, or about 40-50 seconds, or about 45 seconds. In some instances, DEFINITY-II is activated using a shaking rate of about 4950 rpm and a shaking time of about 45 seconds, and optionally DEFINITY-I is activated using a shaking rate of about 4530 rpm and a shaking time of about 45 seconds. These shaking parameters for DEFINITY-II apply to various teachings provided herein. The methods provided herein therefore facilitate the differentiation of a non-aqueous UCA formulation from aqueous UCA formulations such as DEFINITY®.

Other methods provided herein comprise identifying a labeled vial comprising a UCA formulation requiring activation for a pre-determined period of time using a shaking device comprising a detector and set to the pre-determined period of time or capable of automatically selecting the pre-determined period of time based on the identity of the vial, and activating the UCA formulation to form gas-filled microspheres. The pre-determined period of time may be 45 seconds or it may be 75 seconds, although it is not so limited.

Other methods provided herein comprise differentiating between two or more aqueous UCA formulations (such as for example DEFINITY®, DEFINITY-III and DEFINITY-IV), which require different activation times and optionally different shaking rates. The two or more aqueous UCA formulations may be differentiated based on their fill volume (i.e., the amount of liquid in their respective containers), or based on container shape and size. Fill volumes may be assessed, for example, using optical approaches (e.g., measuring absorbance of light by the formulation at a particular position along the length of the container). Container shape and size may be assessed, for example, using the holder which holds the container. Once an aqueous UCA formulation is identified (through differentiation from other UCA), it may then be activated for its prescribed period of time and using its prescribed shaking rate. Where the methods involve differentiation between two or more UCA formulations, the activation means (e.g., the shaking device) may be set to shake at a pre-determined period of time, or it may be set to shake for two or more different pre-determined periods of time and would therefore be capable of automatically selecting one such period of time. Such means may comprise a detector. Similar methods are provided for differentiating and optionally activating non-aqueous UCA formulations. Similar methods are provided for differentiating between aqueous and non-aqueous UCA formulations, and optionally activating one or both UCA formulations.

Other methods provided herein comprise identifying an aqueous UCA formulation requiring activation for a pre-determined period of time, using a device that distinguishes the aqueous UCA formulation from a non-aqueous UCA formulation, and activating the aqueous UCA formulation for the pre-determined period of time to form gas-filled microspheres.

Other methods provided herein comprise identifying a UCA formulation requiring activation for a pre-determined period of time, using a device that distinguishes a non-aqueous UCA formulation from an aqueous UCA formulation (or vice versa), and activating the UCA formulation for a pre-determined period of time to form gas-filled microspheres. The device may be set to activate for only one pre-determined period of time (e.g., about 45 seconds if an aqueous UCA or about 75 seconds if a non-aqueous UCA), or it may be set to activate for two or more different pre-determined periods of time (e.g., about 45 seconds and about 75 seconds). It is to be understood that where two or more pre-determined periods of time are contemplated, such periods of time are different from each other.

Still other methods are provided that comprise identifying a UCA formulation requiring activation for a pre-determined period of time and/or pre-determined shaking rate, and activating the UCA formulation for the pre-determined period of time and/or rate to form gas-filled microspheres. For example, the methods comprise identifying a non-aqueous UCA formulation requiring activation for a pre-determined rate and time, and activating the non-aqueous UCA formulation for the pre-determined period of time and rate to form gas-filled microspheres. The UCA formulation may be identified and activated using a shaking device set to the pre-determined period of time and/or rate or capable of automatically selecting the pre-determined period of time and/or rate based on the identity of the UCA formulation.

Thus, in some instances, the methods comprise identifying a UCA formulation requiring activation for a pre-determined period of time and/or rate, and activating the UCA formulation for the pre-determined period of time and/or rate to form gas-filled microspheres using a shaking device that is set to two or more pre-determined periods of time or capable of automatically selecting between two pre-determined periods of time based on the identity of the UCA formulation. In some instances, the identity of the UCA formulation is provided by a label or tag on the container (e.g., vial) housing the formulation. In some instances, the identity of the UCA formulation is provided by the formulation itself or its volume, as described herein in more detail. The UCA formulation may be an aqueous UCA formulation or it may be a non-aqueous UCA formulation. The pre-determined period of time may be about 45 seconds. The pre-determined period of time may be in the range of 60-120 seconds or about 75 seconds.

The pre-determined shaking may include at least 1000, at least 2000, at least 3000, at least 4000, at least 4500, at least 5000, at least 6000 or more shaking motions per minute. In some instances, vigorous shaking includes shaking in the range of 4000-4800 shaking motions per minute. For shaking may include 4530 revolutions per minute, or 4077-4756 revolutions per minute. Other shaking rates may be in the range of less than 4830, such as 4530. Other shaking speeds may include shaking in the range of 4830 to 6000 rpm. For example, shaking may include a shaking speed of about 4950. Other shaking speeds in the range of 4000-6000 also may be used in other embodiments.

Alternatively, other methods provided herein comprise identifying a labeled vial comprising a UCA formulation requiring activation for a fixed period of time and a pre-determined shake speed using a shaking device comprising a scanner set to the fixed period of time and pre-determined shake speed or capable of automatically selecting the pre-determined shake speed based on the identity of the vial, and activating the UCA formulation to form gas-filled microspheres. The pre-determined shake speed may be 4530 rpm in some embodiments.

Still other methods comprise activating a UCA formulation using a shaking device that identifies the UCA formulation and automatically selects an activation time or shake speed (or shake rate, and the terms are used interchangeably herein) or both based thereon, wherein the UCA formulation is identified based on a unique identifier other than shape or size of a vial housing the UCA formulation.

Other methods comprise activating a first UCA formulation using a shaking device that can distinguish a first vial comprising the first UCA formulation from a second vial comprising a second UCA formulation.

Yet other methods comprise identifying a labeled vial comprising an aqueous UCA formulation requiring activation for a pre-determined first period of time, using a shaking device comprising a scanner and set to the pre-determined period of time or capable of automatically selecting the first pre-determined period of time from two pre-determined periods of time, based on the identity of the vial, and activating the UCA formulation to form gas-filled microspheres.

All of these methods may be automated in whole or in part. In some instances, the devices first identify the vial containing the UCA formulation and provide a prompt to the user to confirm the identification. In other instances, the devices identify and activate without any user input.

### Devices

Identification of a UCA formulation and/or distinction between different UCA formulations can be achieved in a number of ways. For example, devices may be used with scanners able to read labels on the UCA formulation container (e.g., vial). In other instances, identification and/or distinction between different UCA formulations can be achieved using devices that recognize the shape and size of a container housing an aqueous UCA formulation versus a container housing a non-aqueous UCA formulation. These latter devices may comprise a single holder or they may comprise two or more holders. If a single holder, the holder may be capable of holding a container (e.g., a vial) housing a non-aqueous UCA formulation and incapable of holding a container (e.g., vial) housing an aqueous UCA formulation. Alternatively, the holder may be capable of holding a container (e.g., a vial) housing an aqueous UCA formulation and incapable of holding a container (e.g., vial) housing a non-aqueous UCA formulation.

According to one aspect, a device receives a container holding a UCA formulation, detects the UCA formulation type and performs different actions depending on the type of UCA formulation that is detected. The device associates certain actions with each UCA formulation type. After detecting a certain UCA formulation type, the device automatically performs the actions associated with that UCA formulation type.

A variety of different actions can be performed based on the UCA formulation type that is detected. In some embodiments, the device shakes the sample. In some embodiments, the device performs a specific shaking duration, pattern, and/or rate depending on the sample type that is detected. Examples of different shaking patterns include but are not limited to: side to side reciprocation, up and down reciprocation, vibration, a spinning motion, a figure-eight path, a circular path and back-and-forth tilting (e.g. rotating the container by some angle and reversing the action). For example, in one illustrative embodiment, the device associates a shaking duration of about 45 seconds with sample type “A” and about 75 seconds with sample type “B.” The shaking rate may be the same for both sample type “A” and sample type “B” (e.g., about 4530 rpm). When the device detects a sample type “A,” the device automatically shakes the sample for about 45 seconds without requiring the user to input a shaking time. When the device detects a sample type “B,” the device automatically shakes the sample for about 75 seconds. As another example, in one illustrative embodiment, the device associates a shaking rate of about 4530 rpm with sample type “A” and about 4950 rpm with sample type “B.” The shaking time may be the same for both sample type “A” and sample type “B” (e.g., about 45 seconds). When the device detects a sample type “A,” the device automatically shakes the sample at about 4530 rpm without requiring the user to input a shaking rate. When the device detects a sample type “B,” the device automatically shakes the sample at about 4950 rpm.

Thus, this disclosure further contemplates devices that are capable of varying one or more parameters upon identification (and thus differentiation) of sample types. As an example, one device may shake with the same pattern and at the same shaking rate for all sample types, but may shake different sample types for different durations (i.e., different shaking times). As another example, one device may shake with the same pattern and for the same time for all sample types, but may shake different sample types at different rates (i.e., different shaking rates). As yet another example, one device may shake with the same shaking rate and for the same time for all sample types, but may shake different sample types with different shaking patterns. Alternatively, the device may respond to each sample type identified by setting, including potentially altering, two parameters, such as shaking rate and shaking time, or shaking rate and shaking pattern, or shaking time and shaking pattern. In still another embodiment, the device may respond to each sample identified by setting, including potentially altering, all three of these parameters (i.e., shaking rate, shaking time, and shaking pattern).

It should be appreciated that many other actions can be associated with a sample type. Examples of different actions that a device can perform in reaction to a detected sample type include but are not limited to: adjusting temperature settings, adjusting humidity settings, adjusting light settings (e.g. subjecting the sample to different intensities and/or frequencies of light or sound), and/or inputting different substances into the container (e.g. reagents, dyes or other suitable additives).

This disclosure also contemplates devices that may shield outside interference that may affect operation of the device. For example, external interferences (e.g., RFID signals) may interfere with messaging on the display screen, may interfere with reading of an indicator on the container by a detector (e.g., an antenna), or may interfere with another operation of the device. In view of the above, devices described herein may include one or more features to shield outside interferences. For example, in some embodiments, the devices may include one or more patches (e.g., stickers, labels, or tape) applied to areas of the housing needing shielding. For example, one or more patches may be affixed in regions near the detectors of the device. In another example, at least a portion of the housing may be internally coated (e.g., sprayed) with a material that blocks such interference. In some embodiments the entire inner surface of the housing may be coated with a material that blocks such interferences. For example, a PPG PN or spraylet material may be used to coat the housing. In some embodiments, the cover of the housing also may be sprayed. In still another embodiment, the housing, or at least a portion of the housing, may be formed of a material arranged to block interferences.

### Indicators

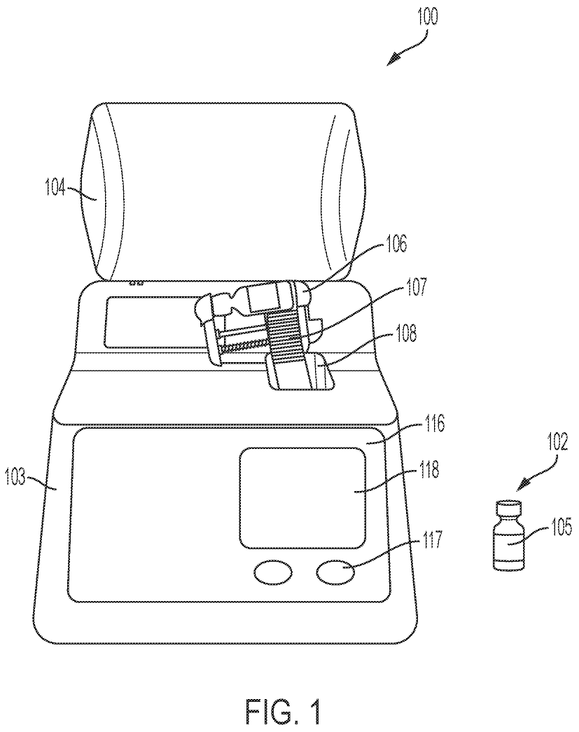

In some embodiments, the container holding the sample includes an indicator that indicates the sample type and the device include a detector that reads the indicator to detect the sample type. For example, as shown in FIG. 1, a container 102 holding a sample to be activated via a device 100 may include an indicator 105 that is machine- or device readable. Examples of machine- or device-readable indicators include magnetic stripes, chips/microchips, barcodes including linear, matrix and 2D barcodes, radio frequency identification (RFID) tags, color labels that are identifiable by color detection, and the like. Barcodes such as linear barcodes may be those that comply with or meet Uniform Code Council standards or Health Industry Business Communications Council standards. Such indicators may in turn be read, for example, from a device such as a magnetic stripe reader, a chip reader, a barcode scanner or reader, an RFID tag reader, and the like. Virtually any labeling technology that has been used for authentication and/or “track and trace” purposes may be used in conjunction with the containers provided herein.

The indicator may be positioned on any suitable portion of the sample container, such as the body of the container or the cap. In some embodiments, the indicator is integrally formed with or otherwise a part of the sample container. For example, the indicator may be a colored cap or a physical feature such as a protrusion or an indentation on the sample container. In other embodiments, the indicator is attached to the container via, for example, adhesive, magnets, hook-and-loop type fasteners, mechanical arrangement such as sliding the indicator behind holding tabs, or any other suitable attachment arrangement.

The indicator may provide the end user or an intermediate handler of the container a variety of information including but not limited to source and/or producer of the formulation contained therein, including for example the name of the company or company subsidiary that made the formulation and/or that produced components of the formulation, the date on which the formulation was made, the physical location where the formulation was made, the date of shipment of the container, the treatment of the container including for example whether it was stored in a remote location and the conditions and length of such storage, the date on which the container was delivered, the means of delivery, the National Drug Code (NDC) as prescribed by the FDA, content of the container, dose and method of use including route of administration, etc.

The indicator may serve one or more purposes including for example authentication of the container and the formulation contained therein. Authentication means the ability to identify or mark the container as originating and having been made by an authorized party, and it allows an end user or other party to identify containers and formulations originating from another, unauthorized party. The indicator may also be used to track and trace a container. This feature may be used to follow a container and the formulation contained therein following production and up to the point of administration to a subject. In this regard, the movement of the container during that period of time may be stored in a database, and optionally such a database may be accessible to an end user to ensure the integrity of the formulation.

The indicator may also be a combined indicator, intending that it may contain information that is read using two different modes. For example, the indicator may contain information that is apparent and understandable to the visible eye (e.g., it may recite the name of the producer in words) and other information that is machine-readable, such as RFID embedded or barcode embedded information.

The indicator may also be a dual use indicator, intending that it may serve two or more purposes. For example, the indicator may contain information that identifies the formulation and further information that identifies the manufacturer and/or date of manufacture. This information may be conveyed in the same format or using different format (e.g., one may be provided in an RFID indicator and the other may be provided in a barcode label).

The label may also be capable of having information recorded on it (e.g. using RFID technology) by the device used to shake the vial. For example, such information may be used to prevent re-activation of the vial by any appropriately equipped device if it has previously been shaken and is now beyond the expiry period for re-activation of previously-activated containers.

The indicator may provide content that is visible and understandable to a human, such as for example the name of the manufacturer. Alternatively or additionally, the indicator may contain information that while readily visible to the human eye nevertheless provides no meaningful information in the absence of a look-up-table or other form of database to which reference must be made. Such information for example may be provided as alpha-numeric code.

In some embodiments, the UCA formulation is in a container, such as a vial, and such container is labeled. The container may have an indicator in the form of a label that is affixed to one or more of its outer surfaces. In some embodiments, the indicator is a paper label or other such label that is visible by eye and capable of being read and understood by an end user without further aid or device. Alternatively, as discussed above, the indicator is one that is machine or device readable.

### Detectors and Container Holders

The device may include any suitable detector for reading the indicator. In some embodiments, the detector may operate via visual, photographic, imaging, electromagnetic, visible light, infrared and/or ultraviolet modalities.

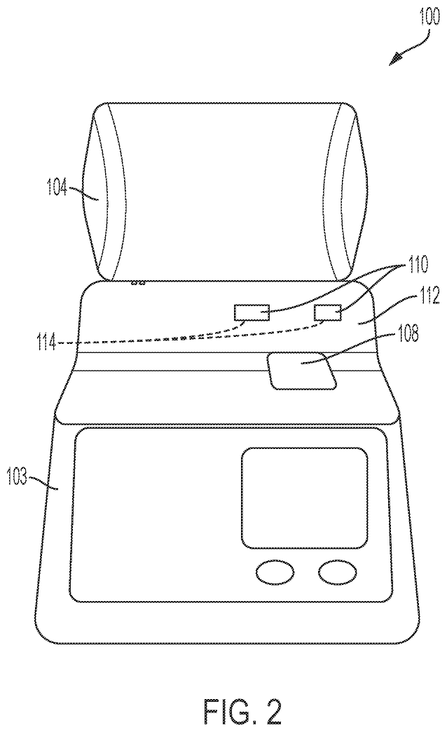

FIGS. 1 and 2 depict illustrative schematics representing a device 100 having a detector for reading an indicator from a sample container 102. As shown in these views, the device 100 includes a housing 103, also referred to herein as a base, and a cover 104. In some embodiments, the cover is opened by rotationally pivoting the cover relative to the housing, although the cover may be attached to the housing in other suitable manners (e.g., slidable relative to the base). In some embodiments, the cover may be entirely lifted off and removed from the housing. The sample container 102 to be used with the device includes an indicator 105, as described above, that indicates the type of sample in the container.

In some embodiments (see, e.g., FIG. 1), the device includes a holder 106 arranged to receive and hold the sample container. In some embodiments, the holder is attached to a shaker arm 107 that extends through an opening 108 in the housing. The shaker arm may be connected to a shaking means, as will be described, arranged to move the shaker arm to activate the UCA formulation. For example, the shaker arm may be moved in a figure of eight path to shake the container and activate the UCA formulation.

In some embodiments, as shown in FIG. 2, the device may include one or more detectors 110 for reading the indicator. For example, the device may include first and second detectors for reading the indicator. In some embodiments, the detectors may be attached to a first, upwardly-extending wall 112 of the housing, with the detectors being aligned with, or located at or near a plane extending through, the first and second lateral sides of the opening 108 through which the shaker arm extends (see FIG. 1). As will be appreciated, the upwardly extending wall may be angled relative to the wall in which the opening is formed. The one or more detectors may have other suitable arrangements in other embodiments. For example, the detector(s) may be attached to the wall through which the opening is formed, such as on either side of the opening. A first detector also may be attached to the wall in which the opening is formed while a second detector is attached to the upwardly extending wall.

In some embodiments, the first and second detectors may include first and second antennas. In such embodiments, the antennas may be arranged to receive information from (e.g., read) one or more indicators on the container. For example, the antenna may read RFID signals from an RFID indicator. In some embodiments, such as where the indicator is configured to both send and receive information, the antennas also may be arranged to transmit information back to the indicator. For example, the antenna may supply the date and time of the shaking to the indicator such that the container may not be activated a second time.

In some embodiments, the detectors are arranged such that the detectors may read the indicator no matter where the holder is located when the container is inserted in the holder. For example, in some embodiments, the holder may travel in a substantially figure-of-eight path, with the holder stopping at different positions relative to the opening and to the housing during activation of the UCA formulation. In such embodiments, the detectors (e.g., antennas) are arranged such that the detectors (e.g., antennas) may read the indicator no matter where along the figure of eight path the holder is positioned when the next container is inserted into the holder.

In some embodiments, the indicator may include a barcode and the detector is a barcode scanner. In some embodiments, the indicator is an RFID tag and the detector is an RFID reader. In some embodiments, the indicator is a colored label and the detector is a color detecting scanner. In some embodiments, the indicator is a chip/microchip and the detector is a chip/microchip reader.

In some embodiments, the device may include one or more of the same types of detectors. For example, the device may include two RFID readers to read an RFID tag on the container. The device also may include two different detectors. For example, the device may include an RFID reader and a chip/microchip reader. As will be appreciated, the device may include more than two detectors in other embodiments.

In some embodiments, the detector(s), e.g., an RFID reader, is connected to the device via one or more wires 114. The detector(s) also may be wirelessly connected to the device. In some embodiments, the detectors transmit information regarding the UCA formulation to the device. For example, the detectors may transmit the identity of the UCA formulation. The detectors also may transmit one or more activation parameters (e.g., shaking time and/or speed(s)) provided on the indicator.

In some embodiments, the sample containers may include an indexing feature that ensures that the indicator on the container is properly aligned with the detector on the device. Examples of indexing features include physical recesses or protrusions on the container cap or body that align with corresponding features on the holder such that the container can only fit into the holder in one orientation.

In some embodiments, speed info may be present on the same indicator as the UCA formulation identity information or it may be present on another indicator. In some embodiments, the detector includes means for reading the two sets of info, which may be the same or may be different.

In some embodiments, the indicator is a physical component, such as a protrusion or an indentation on the container. The detector may be a button on the device that is pushed or a sensor that is otherwise activated due to physical interaction with the physical component. In one illustrative embodiment, the indicator is a specifically shaped protruding tab on the cap of the sample container, and the device includes corresponding slots into which the tabs can be inserted. Each sample type may be associated with a specific tab shape, and each tab shape exclusively fits with only one of the slots on the device. For example, an L-shaped tab may be associated with sample type “A” while an oval-shaped tab may be associated with sample type “B.” The portion of the device that interacts with the container cap has associated slots; one that receives the L-shaped tab and one that receives the oval-shaped tab. When an L-shaped tab is inserted into the holder, the tab presses a button within the L-shaped slot, and the device knows a sample type “A” has been received. When an oval-shaped tab is inserted into the holder, the tab presses a button within the oval-shaped slot, and the device knows a sample type “B” has been received.

In some embodiments, the device may detect the sample type based on one or more properties of the sample container. Examples of properties include weight, optical properties, and size of the container. Regarding weight, the weight of the sample may reflect a sample type. For example, containers having samples of type A may have one weight range and containers having samples of type B may have a second, different weight range. The device may include a scale or other weight detection apparatus that determines the combined weight of the container and sample. If the weight falls within the first range, the device determines that the sample is type A and if the weight falls within the second range, the device determines that the sample is type B. The weight detection apparatus may be integrated into the holder or may be a separate weighing station on the device. In the case of a separate weighing station, the user places the container in/on the weight detection apparatus, the device measures the weight to detect the sample type, and then the user or the device itself moves the sample container to the holder.

Regarding optical properties, each sample type may be associated with a known optical property. Examples of optical properties include but are not limited to index of refraction, absorption, and fluorescence. In some embodiments, the device may include a suitable instrument for measuring the optical property and, from the measurement, determine the associated sample type.

Regarding sample container size, each sample type may be associated with a different sized container. For example, sample type “A” may have a container that is larger than the container used for sample type “B.” The device may detect container size in variety of ways. In some embodiments, the device has more than one holder—each holder being sized to accommodate one of the sample container sizes. For example, each sample container size may only fit into one of the holders. The device detects when and which holder has received a container. By knowing which holder has a container, the device determines the sample container size and the sample type associated with that container size. In another embodiment, the device has a single holder that can accommodate differently sized containers. For example, the holder may have a spring-biased end that can be moved to different positions to accommodate larger containers. The device may have buttons or other sensors that detect the receipt of a container and the size at which the holder has been enlarged to in order to determine the container size. As another example, the user may need to manually adjust the holder size by removing filler pieces, flipping open doors, or otherwise moving components to size the holder to appropriately and snugly accommodate the sample container. The device would then sense the size of the holder and determine the container size accordingly. In other embodiments, the device may include visual detectors such as a camera and/or a laser to detect the size of the container. For example, a camera may take an image of the container and process the image to determine the size of the container. As another example, a laser may be directed to a position that would hit the container if a large-sized container is used but pass through nothing if a small-sized container is used, and the device would accordingly detect the container size by determine whether the laser had been obstructed or otherwise interfered with along its path.

In some embodiments, the device may be arranged to detect whether the sample is expired (e.g. by reading information from an indicator on the sample container). The device may alert the user of this and/or may prevent the device from operating while the expired sample is received by the device.

In some embodiments, the device may include a display that can communicate a variety of different messages to a user. For example, the display may indicate the status of the device, errors, sample type, and may alert the user to any potential problems. In some embodiments, as shown in FIG. 1, the device may include a control panel 116, which includes control buttons 117 and a display 118. In some embodiments, the user alerts may be displayed on the display 118.

The alerts also may be sent to a mobile device associated with the user. For example, in some embodiments, the device may be connected to a computer or network, e.g. via Wi-Fi, USB, or other connection. This connection may be used to remotely maintain the device, e.g. patching/upgrading software and/or monitoring the device status and usage. The connection may also be used for data delivery, e.g., data obtained by the device may be sent to a database and/or printer and/or to a user.

It should be appreciated that the device may have a variety of different features to aid in operation. In some embodiments, the device may include a counting feature that can track how many times the machine has been used to conduct certain actions. Alternatively, a counting device may track the number of revolutions/oscillations the shaking device has performed. Such a feature may be used for maintenance anticipation and monitoring of device performance. The counter may be digital or manual. In some embodiments, the counter may be used to track how many times a specific sample has been acted upon, e.g., the counter may track how many times a specific container, also referred to herein as a vial, has been activated. In some embodiments, the counter may be used to generally track how many of each type of sample has been received and acted upon.

Alerts may be auditory and/or visual. Examples of alerts include: alerting the user that a specific action has been performed on a sample a certain number of times, that an action has not been performed adequately or has been performed too much (e.g. shaking time was too long or too short), that the cover is open, that the container is not seated appropriately in the holder, and/or that the device requires or is soon to require maintenance. In some embodiments, the device will alert a user that the action that has been performed on the sample or container (e.g., vial) exceeded or is near the limits of an acceptable range. For example, the device may alert the user if the device performance exceeds or is near the limits on acceptable ranges for the rate or duration of shaking. As an illustrative example, the device may have shaken the sample at a rate that was too high, too low, or close to the upper or lower limit on shaking rate. The device would alert the user of this potential concern.

In some embodiments, the device includes an indicator portion separate from the display that indicates to a user the sample type that has been detected. The indicator may have lights that indicate sample type (e.g., aqueous or non-aqueous UCA formulation), or may have a display that displays the name of the sample type.

In some embodiments, while a user need not enter the sample type and/or the specific action to be taken, the device may ask the user to confirm the sample type that has been detected before the device can act on the sample.

In some embodiments, the holder includes a button or other sensor to detect whether a container has been appropriately received. In some cases, the device will not operate unless it detects a container in the holder.

In some embodiments, the device may include a button or other sensor to detect whether the cover is appropriately closed. In some cases, the device will not operate unless the cover is in a closed position. For example, in some embodiments, the housing may include a magnetic reed switch arranged to detect a proximity of a magnetic attached to the housing. As It will be appreciated that other suitable sensors may be used to detect the presence of the cover. For example, in other embodiments, the cover may include a protrusion that is received in a corresponding opening in the housing when the cover is in a closed position.

In some embodiments, the device may record and transmit information such as vial usage, shaking times, temperature and other conditions, device usage, analysis results, to a database or other data storage location. In some embodiments, information from the device may be compared with databases of information to detect abnormalities with the device or the sample, and/or the comparisons may be used to categorize the sample. In some embodiments, once the indicator has been read, the device may operate a program in the device to activate the UCA formulation. In other embodiments, the device may take one or more parameters stored on the indicator (e.g., speed, shaking rate) and activate the UCA formulation via the parameters.

In some embodiments, the device may count and monitor the number of samples processed and/or the condition of the device and accordingly advise the user of a need to reorder items such as samples, device parts that require replacement, etc.

In some embodiments, the sample holder is arranged to accommodate only a single container size and does not allow a larger container to fit. The holder may have a cap cover arranged to hold the vial in place. For example, the cap cover may receive the cap of the vial and hold the vial via interference fit, a threaded arrangement (e.g. outer threads on the vial cap that mate with inner threads on the cap cover 21), mechanical interlock or any other suitable arrangement. In some embodiments, the sample holder may have a spring at the base of the holder to keep the vial from moving within the holder and partially eject the vial once the cap is removed for ease of removing the vial. In other embodiments, the holder can expand to accommodate a larger container, and also detect the holder size to detect the sample type, as previously discussed.

In some embodiments, the holder, or at least a part of the holder is attachable to the shaker arm. In other embodiments, the holder, or at least a portion of the holder may be integrally formed with the shaker arm. In some embodiments, at least a portion of the shaker arm may be arranged to hold a portion of the container.

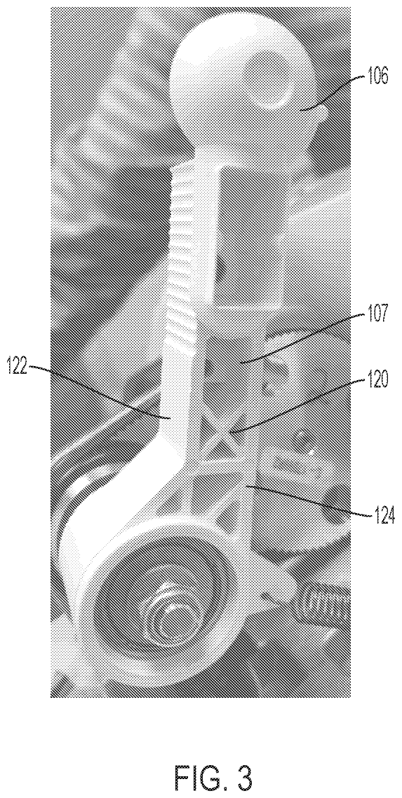

In some embodiments, as shown in FIG. 3, the shaker arm may include one or more stiffening ribs 120. In some embodiments, the stiffening ribs may extend between first and second (e.g. front and rear) walls of the shaker arm. In some embodiments, the stiffening rib(s) may extend only part way between the first and second walls of the shaker arm. The stiffening rib(s) may intersect another stiffening rib(s) in some embodiments. In some embodiments, the stiffening ribs may extend substantially perpendicular to one or both of the first and second walls of the shaker arm. The stiffening rib(s) also may extend substantially parallel to one or both of the first and second walls of the shaker arm. It will be appreciated, that that the stiffening ribs may be the same shape and size in some embodiments, although the shape and size of the stiffening ribs may vary from rib to rib. In some embodiments, each rib may have the same thickness, although the thickness may vary from rib to rib. One or more ribs also may have a thickness that varies. For example, the thickness of a rib may taper in some embodiments.

In some embodiments, the arm may be formed of a material, or combination of materials, arranged to resist thermal degradation during shaking. In some embodiments, the material or combination of materials may include a maximum long run temperature of between 200° F. and 500° F. In some embodiments, the material or combination of materials may include a tensile strength of between about 6000 psi and about 41000 psi. In some embodiments, the shaker arm may be formed of Ultradur B, Nylon 6, PET polyester, PEI (Iltem), Nylon 4-6 Stanyl, PPA (Amodel), PBT, PES, polymide (aurum), PPS polyphenyl sulfide, PEEK, and/or combinations thereof.

### Containers (e.g., Vials)

The UCA formulations may be provided in a container (or housing). In some embodiments, the container is a vial. The vial may be made of any material including but not limited to glass or plastic. The glass may be pharmaceutical grade glass. The container may be sealed with a stopper such as a rubber stopper. In some embodiments, the container is a 0.5-10 mL container. The container may be a 1-5 mL container, or a 1 or 2 mL container.

Such volumes refer to the volume of liquid typically placed into the container (referred to as the liquid fill volume). This is in contrast to the entire internal volume of the container, which will be higher than the liquid fill volume. Examples of liquid fill and internal volumes are as follows: Schott 2 mL (liquid fill volume) vial having a 2.9 mL internal volume; Schott 3 mL (liquid fill volume) vial having a 4.5 mL internal volume; and Wheaton 1 mL (liquid fill volume) v-vial having a 1.2 mL internal volume.

As will be understood in the context of this disclosure, the internal volume of a container may be occupied with lipid formulation and gas. An example of a suitable container is the Wheaton 2 ml glass vial (commercially available from, for example, Nipro, Cat. No. 2702, B33BA, 2 cc, 13 mm, Type I, flint tubing vial), having an actual internal volume of about 3.75 ml. An example of a suitable stopper is a West gray butyl lyo, siliconized stopper (Cat. No. V50, 4416/50, 13 mm, WS-842). An example of a suitable seal is a West flip-off aluminum seal (Cat. No. 3766, white, 13 mm, 13-F-A-591). The containers are preferably sterile and/or are sterilized after introduction of the lipid solution and/or gas as described in published PCT application WO99/36104.

In some embodiments, the container is a flat bottom container such as a flat-bottom vial. Suitable vials include flat bottom borosilicate vials, including Wheaton vials. In some embodiments, the container is a non-flat bottom container or vial. In some embodiments, the container is a V-bottom container such as a V-bottom vial. In some embodiments, the container is a round-bottom container such as round-bottom vial. In some embodiments, the container has converging walls such that its bottom surface area (or bottom surface diameter) is smaller than its top (opening) surface area (or diameter) or smaller than any diameter therebetween (e.g., a body diameter). For clarity, a V-bottom container or vial has converging walls, and its bottom surface area is significantly smaller than any of its top or body surface areas.

It is to be understood that although some of the embodiments described herein refer to vials, they are to be read more broadly to encompass any suitable container, unless explicitly stated otherwise.

### The Shaking Means

As discussed above, the device is arranged to shake a container holing a UCA formulation to activate the UCA formulation. Examples of shake speeds for DEFINITY® and DEFINITY-II are shown below in Tables 1 and 2. As will be appreciated in view of the above, the activation times and shake speeds to achieve optimal microsphere number and equivalent diameter were different for the two products. In general, increasing the shake speed decreased the time. The longer shake time required for DEFINITY-II compared to DEFINITY® could be overcome by a small increase in the shake speed. The DEFINITY® shake time could be decreased by increasing the shake speed.

TABLE 1
DEFINITY-II

| RPM | Activation Time (sec) | Diameter (μm) | Counts/mL × 10⁹ |
|---|---|---|---|
| 4900 | 120 | 1.47 | 5.54 |
| 5200 | 60 | 1.47 | 5.87 |
| 5300 | 45 | 1.61 | 4.90 |
| 5400 | 45 | 1.43 | 5.99 |
| 5500 | 35 | 1.30 | 5.83 |

TABLE 2
DEFINITY ®

| RPM | Activation Time (sec) | Diameter (μm) | Counts/mL × 10⁹ |
|---|---|---|---|
| 4300 | 45 | 1.45 | 3.39 |
| 4800 | 25 | 1.50 | 2.50 |

As also discussed above, the inventors have recognized that the shaking means may experience slippage and stray resonance, which may affect the device's ability to achieve consistent activation over a broad range of speeds. The inventors have also discovered that providing a shaking means with features that creates friction or provides resistance to movement may minimize or even reduce slippage.

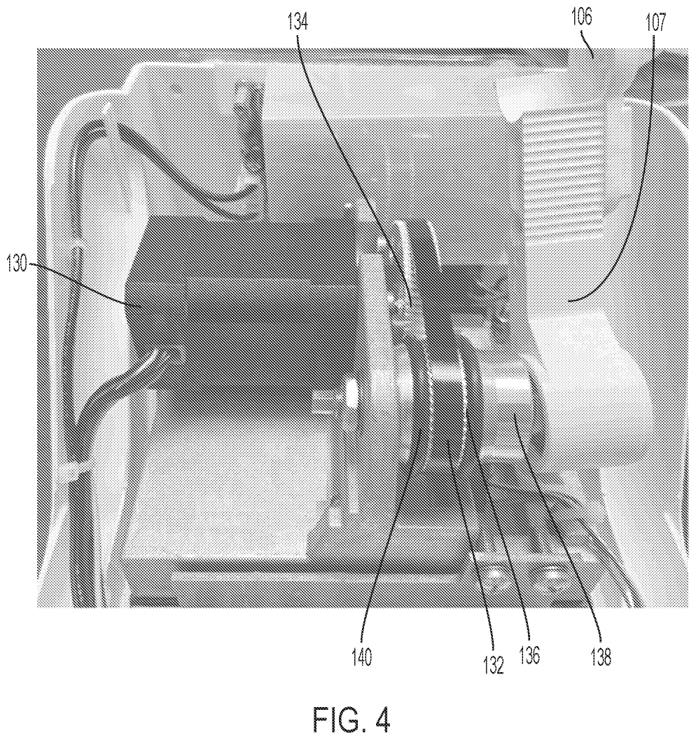

Turning to the figures, FIGS. 4-6 illustrate an example of a shaking means arranged to shake a container to activate the UCA formulation. As shown in these views, the shaking means may include a motor 130 that drives movement of a shaker arm 107, which is configured to hold the container.

In some embodiments, as shown in FIGS. 4-6, the motor may include a direct current (DC) motor. For example, in some embodiments, the shaking means may include a brushless DC motor. In some embodiments, such a motor may allow the device to consistently shake the container over a broad range of speeds. For example, the DC motor may allow the device to operate consistently at high speeds for multiple activations and/or for one or more speeds during the same activation cycle. As will be appreciated, although shown and described as having a DC motor, the shaking means may use other suitable motors, such as an alternating current (AC) motor, to drive movement of the shaker arm.

As shown in FIGS. 4-6, the motor may be connected to the shaker arm via a transmission. In some embodiments, as shown in these views, the transmission may include a gear having first and second toothed wheels 134, 136 and a tooth belt 132. For example, the tooth belt may extend around the gear, such as around at least part of each of the first and second toothed wheels. In such embodiments, teeth on the toothed belt may engage with the corresponding teeth in the toothed wheels. In some embodiments, engagement between the teeth of the tooth belt and the teeth in the corresponding toothed wheels provides resistance to slippage of the toothed belt relative to the gear, and/or to the gears relative to one another.

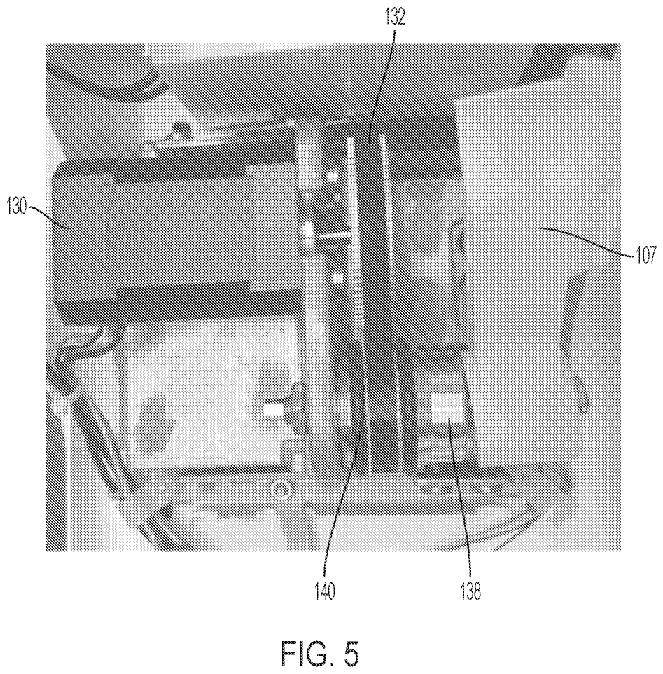

In the embodiment shown in FIGS. 4 and 5, the first toothed wheel 134 is attachable to a portion of the motor, while the second toothed wheel 136 is attached to the shaker arm. In this regard, the tooth belt may be operably coupled to the shaker arm and to the motor such that when the DC motor engages with the tooth belt, the tooth belt, in turn, engages the shaker arm. In such embodiments, movement of the tooth belt drives movement of the holder, which activates the UCA formulation. For example, the motor may cause the first toothed wheel to rotate, moving the tooth belt, which, in turn, causes the second toothed wheel to rotate. In such an example, rotation of the second toothed wheel causes the shaker arm to move (e.g., via a spindle).

As shown in these views, the second toothed wheel may be attachable to a spindle 138, which is attached to the shaker arm 108, such as via a screw. For example, the shaker arm may be placed around an outer surface of the spindle in some embodiments, and then secured to the spindle. The second toothed wheel may be attachable to the shaker arm via other suitable arrangements. For example, in some embodiments, the second toothed wheel may be directly attachable to the shaker arm. In such an example, the shaker arm may have a shaft onto which the second toothed wheel is disposed.

Although the gear includes first and second wheels around which the tooth belt extends in some embodiments, in in other embodiments, the gear may include only one wheel or may include more than two wheels. For example, in other embodiments, the gear may include a single, larger, wheel, which is connected to each of the holder and the motor and around which the tooth belt extends.

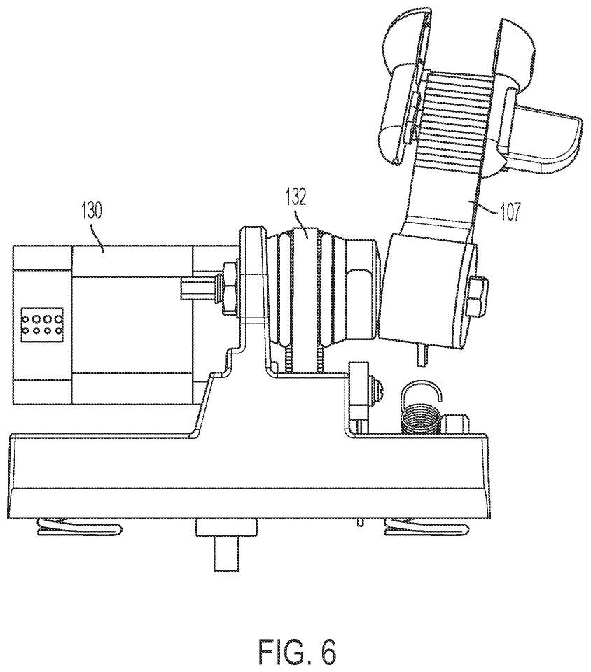

In some embodiments, as shown in FIGS. 4 and 6, the first and second toothed wheels may extend substantially parallel to one another, and to the toothed belt. For example, the first and second toothed wheels, and/or the toothed belt may be about 99%, about 95% and/or about 90% aligned with one another. In such embodiments, the first and second toothed wheels also may be substantially co-planar with one another. For example, the first and second toothed wheels may be about 99%, about 95% and/or about 90% co-planar with one another. In some embodiments, the spindle extends substantially perpendicular to a longitudinal axis of each of the first and second toothed wheels, and to the tooth belt. In some embodiments, as shown in FIG. 6, the shaker arm is angled relative to the first and second toothed wheels. For example, a longitudinal axis may be angled relative to the longitudinal axis of the first and second toothed wheels.

In some embodiments, the transmission also may include first and second O-rings 140. In some embodiments, the O-rings are placed on either side of the second toothed wheel 136. In some embodiments, the O-rings may prevent the second toothed wheel and/or the tooth belt from moving relative to the spindle. For example, the first and second O-rings may prevent slippage of the toothed wheel, and tooth belt, relative to the spindle.

Although O-rings are only shown for preventing slippage of the second toothed wheel and/or toothed belt in these views, it will be appreciated that O-rings also may be used to prevent slippage of the first toothed wheel relative to the motor. For example, in other embodiments, one or more O-rings may be placed around the first toothed wheel.

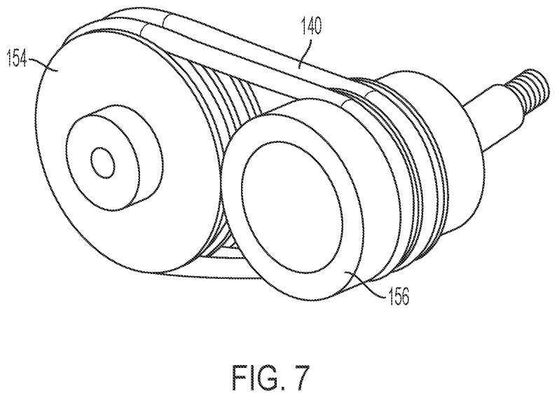

The transmission may have other suitable arrangements for connecting the motor to the shaker. For example, as shown in FIG. 7, the transmission may include two O-rings placed around first and second pulleys 154, 156. As will be appreciated in view of the above, the first pulley may be attachable to the motor while the second pulley is attachable to the shaker arm. In use, movement of the motor may drive movement of the shaker arm via movement of O-rings, and in turn the pulleys.

In such embodiments, as shown in this view, each O-ring may be disposed in a channel formed in each of the pulleys. In some embodiments, each channel may have one or more frictional surfaces that engage with the corresponding O-rings to minimize movement of each O-ring relative to the pulleys. The O-rings also may include one or more frictional surfaces for engaging with the pulleys.

Although two O-rings are shown for connecting the pulleys in FIG. 7, the transmission may use more O-rings in other embodiments. For example, the transmission may include three or more O-rings disposed around the pulleys. In such embodiments, each pulley may include three or more channels for receiving the corresponding O-rings.

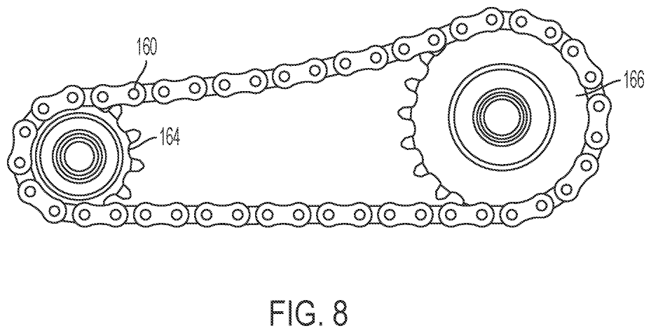

In still another embodiment, as shown in FIG. 8, the transmission may include a drive belt disposable around first and second sprockets 164, 166. As with the above, the first socket may be attachable to the motor while second sprocket may be attachable to the shaker arm. In use, movement of the motor may cause the first sprocket to rotate, which causes the drive chain to move and drive rotation of the second socket. Such rotation causes the shaker arm to move, such as via movement of the spindle. As with the tooth belt, the drive chain may engage with the teeth on each sprocket.

In still another embodiment, the transmission may include a gear box. In such embodiments, the gear box may be attachable to each of the motor and the shaker arm (e.g., via the spindle) for driving movement of the shaker arm).

In some embodiments, the device may be powered by plugging into a wall outlet and/or may run on battery power. In some embodiments, the battery is rechargeable.

### Software and Hardware

As discussed above, the device includes a control panel with one or more buttons and a display. For example, the device may include a start button, and a cancel button. The device also may include an indicator with lights, such as lights corresponding to different sample types. In some embodiments, when the device detects a certain sample type, the light corresponding to that sample type lights up. In some embodiments, the device may further include an indicator separate from the display. The indicator may include signals such as lights that indicate the sample type that has been detected. The device may include an indicator to indicate that the cove is appropriately closed and/or that a sample is properly inserted into the device

As discussed above, some embodiments relate to a device configured to perform different actions based on an identification of a container and/or contents of the container. To this end, a device in accordance with some embodiments may include a computer system including at least one processor programmed to perform identification of the container and/or its contents and upon determining the identification, determine appropriate actions to perform based on the identification.

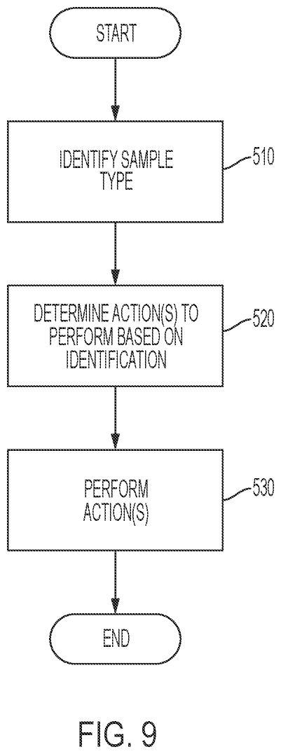

FIG. 9 illustrates a flow chart of a process for selectively performing one or more actions to a vial placed within a device in accordance with some embodiments. In act 510, an identification of a sample type placed in the device is determined. Identification of the sample type may be determined in any suitable way, examples of which are described herein. For example, the device may present a user interface that enables a user to manually enter an identifier of the sample type. Alternatively, the device may be configured to automatically determine the sample type identification by analyzing one or more indicators located on or associated with the vial or by analyzing one or more properties of the contents of the container. In some embodiments, the device may be configured to initially automatically identify the sample type, and if such an automatic identification fails the device may provide an error message and/or prompt a user of the device to manually enter the sample type identification. Embodiments in which the sample type identification is performed manually rather than automatically may enable a less expensive and/or simpler device in which a detector (e.g., an RFID reader, an optical scanner, etc.) is not needed.

In some embodiments, the device may be arranged to accommodate more than one UCA formulation, and step 510 may include identifying the sample. In other embodiments, the device may be arranged to accept only a single UCA formulation, and step 510 may include confirming that the sample matches the UCA formulation of the particular device.

After the sample type has been identified, the process proceeds to act 520, where one or more actions to be performed on the sample are determined. In some embodiments, the device may include at least one storage device configured to store a look-up-table (LUT) or other data structure that stores information about the action(s) to be performed for particular sample type identifications. For example, a first set of actions may be performed if it is determined that the vial contains a first UCA formulation type and a second set of actions may be performed if it is determined that the vial contains a second UCA formulation type. The device may be configured to distinguish between containers with any number of different formulation types or substances contained therein, and embodiments are not limited in this respect.

In some embodiments, act 520 also may include reading one or more actions stored in the indicator on the container. For example, in some embodiments in addition to providing the identity of the sample, the indicator may include one or more process steps, such as the shaking speed and/or time for shaking the sample.

Once the action(s) to be performed are determined, the process proceeds to act 530, where the at least one processor incorporated in the device instructs components of the device to perform the action(s) determined in act 520. In some embodiments, the determination to perform the action(s) may be based, at least in part, on factors other than the identification of the sample type. For example, factors such as whether a lid of the device is closed or whether the device is in a particular operating state may be considered when determining whether to perform the action(s). The at least one processor may communicate with the various components of the device to effectuate the performance of the determined action(s) in any suitable manner.

In some embodiments, the device may be arranged to alert the user and stop performing one or more actions. For example, in some embodiments, the device may be arranged to alert the user and stop shaking if the shake speed is recorded as being too high or too low for the identified sample. In such instances, the device also may be arranged to turn itself off and/or to not complete performance of the action(s) of step 530.

In some embodiments, the device may include a feedback loop such that the action(s) determined in step 520 may be compared to the actions performed in step 530. For example, the shaking speed being performed in step 530 may be compared to the determined speed in step 520. In such embodiments, when the performed action differs from the determined action, the device may alert the user and or terminate the action under step 530.

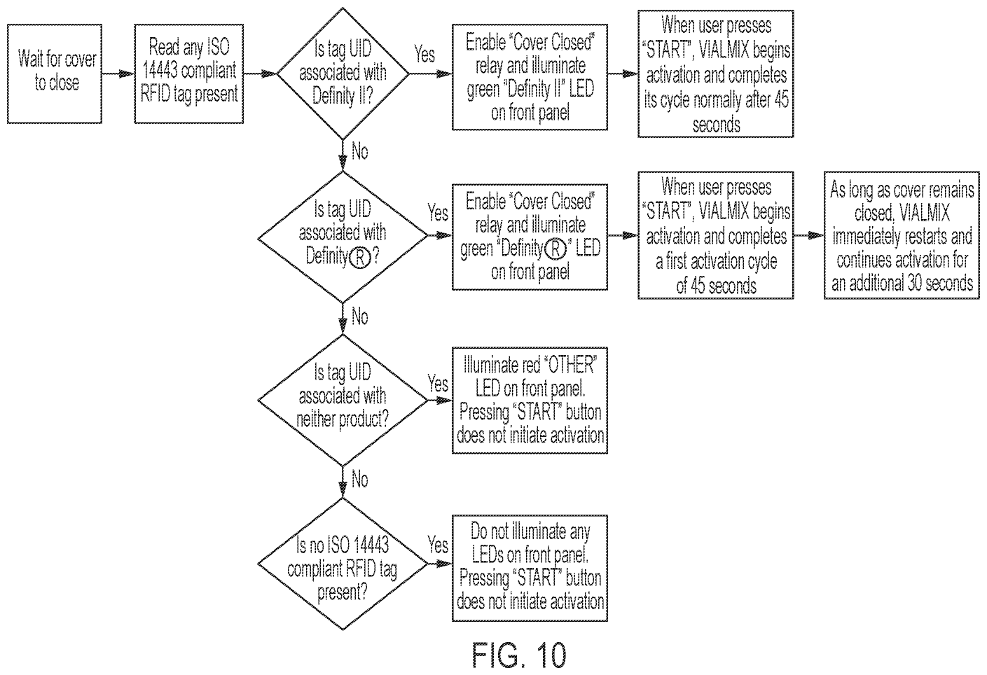

FIG. 10 illustrates a detailed flow chart of a process for determining action(s) to perform on a sample in a vial by using RFID identification in accordance with an embodiment described in more detail below. As illustrated in FIG. 10, if an RFID tag may be present on a container inserted into the device, the device reads the RFID tag to identify the container as containing a first UCA formulation type (DEFINITY®) or a second UCA formulation type (DEFINITY-II). Based on the identification and other conditions being met, the device is activated for a particular amount of time. It is to be understood that, in some embodiments, the device may be configured to initially query whether the container contains DEFINITY-II, followed by another query of whether the container contains DEFINITY, as shown in FIG. 10. Alternatively, in another embodiment, the device may be configured to initially query whether the container contains DEFINITY, followed by another query of whether the container contains DEFINITY-II. In some instances, should the first query yield a positive answer, then the device may not perform the second query.

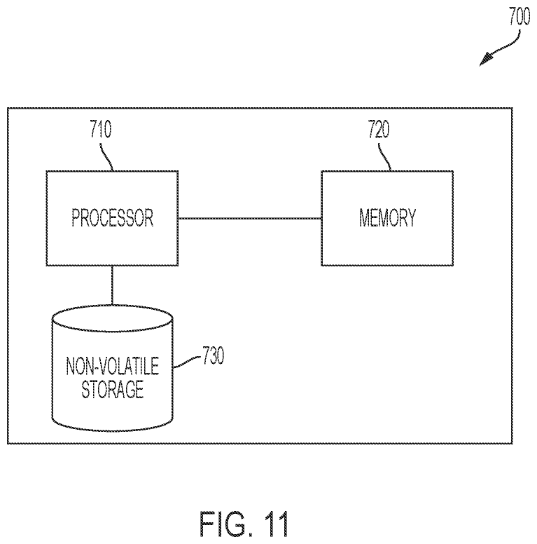

An illustrative implementation of a computer system 700 that may be used in connection with any of the embodiments of the invention described herein is shown in FIG. 11. The computer system 800 may include one or more processors 710 and one or more computer-readable tangible non-transitory storage media (e.g., memory 720, one or more non-volatile storage media 730, or any other suitable storage device). The processor 710 may control writing data to and reading data from the memory 720 and the non-volatile storage device 730 in any suitable manner, as the aspects of the present invention described herein are not limited in this respect. To perform any of the functionality described herein, the processor 710 may execute one or more instructions stored in one or more computer-readable storage media (e.g., the memory 720), which may serve as tangible non-transitory computer-readable storage media storing instructions for execution by the processor 710.

The above-described embodiments of the present invention may be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments of the present invention comprises at least one non-transitory computer-readable storage medium (e.g., a computer memory, a USB drive, a flash memory, a compact disk, a tape, etc.) encoded with a computer program (i.e., a plurality of instructions), which, when executed on a processor, performs the above-discussed functions of the embodiments of the present invention. The computer-readable storage medium can be transportable such that the program stored thereon can be loaded onto any computer resource to implement the aspects of the present invention discussed herein. In addition, it should be appreciated that the reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects of the present invention.

### Uses and Applications

The invention provides methods of use of the UCA formulations provided herein. Once activated, the UCA formulations may be used in vivo in human or non-human subjects or in vitro. The formulations provided herein may be used for diagnostic or therapeutic purposes or for combined diagnostic and therapeutic purposes.

When used as UCA for human subjects, the formulations are activated as described herein in order to form a sufficient number of gas-filled microspheres. The microspheres may be used for diagnostic or therapeutic purposes or for combined diagnostic and therapeutic purposes, optionally in combination with ultrasound. Such microspheres may be used directly (neat) or may be diluted further in a solution, including a pharmaceutically acceptable solution, and administered in one or more bolus injections or by a continuous infusion. Administration is typically intravenous injection. If the microspheres are used in an imaging application, then such imaging is performed shortly thereafter. The imaging application can be directed to the heart or it may involve another region of the body that is susceptible to ultrasound imaging. Imaging may be imaging of one or more organs or regions of the body including without limitation the heart, blood vessels, the cardiovasculature, the liver, the kidneys and the head.

Subjects of the invention include but are not limited to humans and animals. Humans are preferred in some instances. Animals include companion animals such as dogs and cats, and agricultural or prize animals such as but not limited to bulls and horses.

UCAs are administered in effective amounts. An effective amount will be that amount that facilitates or brings about the intended in vivo response and/or application. In the context of an imaging application, such as an ultrasound application, the effective amount may be an amount of lipid microspheres that allow imaging of a subject or a region of a subject.

### Lipids

These UCA formulations comprise one and typically more than one lipid. As used herein, “lipids” or “total lipid” or “combined lipids” means a mixture of lipids.

The lipids may be provided in their individual solid state (e.g., powdered) forms. Alternatively, the lipids may be provided as a lipid blend. Methods of making a lipid blend include those described in U.S. Pat. No. 8,084,056 and published PCT application WO 99/36104. A lipid blend, as used herein, is intended to represent two or more lipids which have been blended resulting in a more homogeneous lipid mixture than might otherwise be attainable by simple mixing of lipids in their individual powdered form. The lipid blend is generally in a powder form. A lipid blend may be made through an aqueous suspension-lyophilization process or an organic solvent dissolution-precipitation process using organic solvents. In the aqueous suspension-lyophilization process, the desired lipids are suspended in water at an elevated temperature and then concentrated by lyophilization.

The organic solvent dissolution method involves the following steps:
(a) Contacting the desired lipids (e.g., DPPA, DPPC, and MPEG5000 DPPE) with a first non-aqueous solvent system. This system is typically a combination of solvents, for example CHCl₃/MeOH, CH₂Cl₂/MeOH, and toluene/MeOH. Preferably, the first non-aqueous solvent is a mixture of toluene and methanol. It may be desirable to warm the lipid solution to a temperature sufficient to achieve complete dissolution. Such a temperature is preferably about 25 to 75° C., more preferably about 35 to 65° C. After dissolution, undissolved foreign matter may be removed by hot-filtration or cooling to room temperature and then filtering. Known methods of filtration may be used (e.g., gravity filtration, vacuum filtration, or pressure filtration).
(b) The solution is then concentrated to a thick gel/semisolid. Concentration is preferably done by vacuum distillation. Other methods of concentrating the solution, such as rotary evaporation, may also be used. The temperature of this step is preferably about 20 to 60° C., more preferably 30 to 50° C.
(c) The thick gel/semisolid is then dispersed in a second non-aqueous solvent. The mixture is slurried, preferably near ambient temperature (e.g., 15-30° C.). Useful second non-aqueous solvents are those that cause the lipids to precipitate from the filtered solution. The second non-aqueous solvent is preferably methyl t-butyl ether (MTBE). Other ethers and alcohols may be used.
(d) The solids produced upon addition of the second non-aqueous solvent are then collected. Preferably the collected solids are washed with another portion of the second non-aqueous solvent (e.g., MTBE). Collection may be performed via vacuum filtration or centrifugation, preferably at ambient temperature. After collection, it is preferred that the solids are dried in vacuo at a temperature of about 20-60° C.

The contents of U.S. Pat. No. 8,084,056 and published PCT application WO 99/36104 relating to the method of generating a lipid blend are incorporated by reference herein.

The organic solvent dissolution-precipitation process is preferred over the aqueous suspension/lyophilization process for a number of reasons as outlined in U.S. Pat. No. 8,084,056 and published PCT application WO 99/36104, including the uniformly distributed lipid solid that results using the organic dissolution method.

Alternatively, the lipids may be provided as individual powders that are dissolved together or individually directly into propylene glycol, glycerol or propylene glycol/glycerol to form the non-aqueous mixture.

As used herein, a lipid solution is a solution comprising a mixture of lipids. Similarly a lipid formulation is a formulation comprising one or more lipids. The lipids may be cationic, anionic or neutral lipids. The lipids may be of either natural, synthetic or semi-synthetic origin, including for example, fatty acids, fluorinated lipids, neutral fats, phosphatides, oils, fluorinated oils, glycolipids, surface active agents (surfactants and fluorosurfactants), aliphatic alcohols, waxes, terpenes and steroids.

At least one of the lipids may be a phospholipid, and thus the lipid blend may be referred to as a phospholipid blend. A phospholipid, as used herein, is a fatty substance containing an oily (hydrophobic) hydrocarbon chain (s) with a polar (hydrophilic) phosphoric head group. Phospholipids are amphiphilic. They spontaneously form boundaries and closed microspheres in aqueous media.

Preferably all of the lipids are phospholipids, preferably 1,2-dipalmitoyl-sn-glycero-3-phosphatidylcholine (DPPC); 1,2-dipalmitoyl-sn-glycero-3-phosphatidic acid (DPPA); and 1,2-dipalmitoyl-sn-glycero-3-phosphatidylethanolamine (DPPE). DPPA and DPPE may be provided as monosodium salt forms.

In some instances, the lipid components may be modified in order to decrease the reactivity of the microsphere with the surrounding environment, including the in vivo environment, thereby extending its half-life. Lipids bearing polymers, such as chitin, hyaluronic acid, polyvinylpyrrolidone or polyethylene glycol (PEG), may also be used for this purpose. Lipids conjugated to PEG are referred to herein as PEGylated lipids. Preferably, the PEGylated lipid is DPPE-PEG or DSPE-PEG.

Conjugation of the lipid to the polymer such as PEG may be accomplished by a variety of bonds or linkages such as but not limited to amide, carbamate, amine, ester, ether, thioether, thioamide, and disulfide (thioester) linkages.

Terminal groups on the PEG may be, but are not limited to, hydroxy-PEG (HO-PEG) (or a reactive derivative thereof), carboxy-PEG (COOH-PEG), methoxy-PEG (MPEG), or another lower alkyl group, e.g., as in iso-propoxyPEG or t-butoxyPEG, amino PEG (NH2PEG) or thiol (SH-PEG).

The molecular weight of PEG may vary from about 500 to about 10000, including from about 1000 to about 7500, and from about 1000 to about 5000. In some important embodiments, the molecular weight of PEG is about 5000. Accordingly, DPPE-PEG5000 or DSPE-PEG5000 refers to DPPE or DSPE having attached thereto a PEG polymer having a molecular weight of about 5000.

The percentage of PEGylated lipids relative to the total amount of lipids in the lipid solution, on a molar basis, is at or between about 2% to about 20%. In various embodiments, the percentage of PEGylated lipids relative to the total amount of lipids is at or between 5 mole percent to about 15 mole percent.

Preferably, the lipids are 1,2-dtpalmttoyl-sn-glycero-3-phosphatidylcholine (DPPC), 1,2-dipalmitoyl-sn-glycero-3-phosphatidic, mono sodium salt (DPPA), and N-(polyethylene glycol 5000 carbamoyl)-1,2-dipalmitoyl-sn-glycero-3-phosphatidylethanolamine, monosodium salt (PEG5000-DPPE). The polyethylene glycol 5000 carbamoyl may be methoxy polyethylene glycol 5000 carbamoyl. In some important embodiments, the lipids may be one, two or all three of DPPA, DPPC and PEG5000-DPPE. PEG5000-DPPE may be MPEG5000-DPPE or HO-PEG5000-DPPE.

A wide variety of lipids, like those described in Unger et al. U.S. Pat. No. 5,469,854, may be used in the present process. Suitable lipids include, for example, fatty acids, lysolipids, fluorinated lipids, phosphocholines, such as those associated with platelet activation factors (PAF) (Avanti Polar Lipids, Alabaster, Ala.), including 1-alkyl-2-acetoyl-sn-glycero 3-phosphocholines, and 1-alkyl-2-hydroxy-sn-glycero 3-phosphocholines; phosphatidylcholine with both saturated and unsaturated lipids, including dioleoylphosphatidylcholine; dimyristoyl-phosphatidylcholine; dipentadecanoylphosphatidylcholine; dilauroylphosphatdylcholine; 1,2-dipalmitoyl-sn-glycero-3-phosphatidylcholine (DPPC); distearoylphosphatidylcholine (DSPC); and diarachidonylphosphatidylcholine (DAPC); phosphatidylethanolamines, such as dioleoyl-phosphatidylethanolamine, 1,2-dipalmitoyl-sn-glycero-3-phosphatidylethanolamine (DPPE) and distearoyl-phosphatidylethanolamine (DSPE); phosphatidylserine; phosphatidylglycerols, including distearoylphosphatidylglycerol (DSPG); phosphatidylinositol; sphingolipids such as sphingomyelin; glycolipids such as ganglioside GM1 and GM2; glucolipids; sulfatides; glycosphingolipids; phosphatidic acids, such as 1,2-dipalmitoyl-sn-glycero-3-phosphatidic acid (DPPA) and distearoylphosphatidic acid (DSPA); palmitic acid; stearic acid; arachidonic acid; and oleic acid.

Other suitable lipids include phosphatidylcholines, such as diolecylphosphatidylcholine, dimyristoylphosphatidylcholine, dipalmitoylphosphatidylcholine (DPPC), and distearoylphosphatidylcholine; phosphatidylethanolamines, such as dipalmitoylphosphatidylethanolamine (DPPE), dioleoylphosphatidylethanolamine and N-succinyl-dioleoylphosphatidylethanolamine; phosphatidylserines; phosphatidyl-glycerols; sphingolipids; glycolipids, such as ganglioside GM1; glucolipids; sulfatides; glycosphingolipids; phosphatidic acids, such as dipalmatoylphosphatidic acid (DPPA); palmitic fatty acids; stearic fatty acids; arachidonic fatty acids; lauric fatty acids; myristic fatty acids; lauroleic fatty acids; physeteric fatty acids; myristoleic fatty acids; palmitoleic fatty acids; petroselinic fatty acids; oleic fatty acids; isolauric fatty acids; isomyristic fatty acids; isopalmitic fatty acids; isostearic fatty acids; cholesterol and cholesterol derivatives, such as cholesterol hemisuccinate, cholesterol sulfate, and cholesteryl-(4′-trimethylammonio)-butanoate; polyoxyethylene fatty acid esters; polyoxyethylene fatty acid alcohols; polyoxyethylene fatty acid alcohol ethers; polyoxyethylated sorbitan fatty acid esters; glycerol polyethylene glycol oxystearate; glycerol polyethylene glycol ricinoleate; ethoxylated soybean sterols; ethoxylated castor oil; polyoxyethylene-polyoxypropylene fatty acid polymers; polyoxyethylene fatty acid stearates; 12-(((7′-diethylaminocoumarin-3-yl)-carbonyl)-methylamino)-octadecanoic acid; N-[12-(((7′-diethylamino-coumarin-3-yl)-carbonyl)-methyl-amino)octadecanoyl]-2-amino-palmitic acid; 1,2-dioleoyl-sn-glycerol; 1,2-dipalmitoyl-sn-3-succinylglycerol; 1,3-dipalmitoyl-2-succinyl-glycerol; and 1-hexadecyl-2-palmitoyl-glycerophosphoethanolamine and palmitoylhomocysteine; lauryltrimethylammonium bromide (lauryl-=dodecyl-); cetyltrimethylammonium bromide (cetryl-=hexadecyl-); myristyltrimethylammonium bromide (myristyl-=tetradecyl-); alkyldimethylbenzylammonium chlorides, such as wherein alkyl is a C.sub.12, C.sub.14 or C.sub.16 alkyl; benzyldimethyldodecylammonium bromide; benzyldimethyldodecylammonium chloride, benzyldimethylhexadecylammonium bromide; benzyldimethylhexadecylammonium chloride; benzyldimethyltetradecylammonium bromide; benzyldimethyltetradecylammonium chloride; cetyldimethylethylammonium bromide; cetyldimethylethylammonium chloride; cetylpyridinium bromide; cetylpyridinium chloride; N-[1-2,3-dioleoyloxy)-propyl]-N,N,N-trimethylammonium chloride (DOTMA); 1,2-dioleoyloxy-3-(trimethylammonio)propane (DOTAP); and 1,2-dioleoyl-e-(4′-trimethylammonio)-butanoyl-sn-glycerol (DOTB).

In some embodiments where DPPA, DPPC and DPPE are used, their molar percentages may be about 77-90 mole % DPPC, about 5-15 mole % DPPA, and about 5-15 mole % DPPE, including DPPE-PEG5000. Preferred ratios of each lipid include those described in the Examples section such as a weight % ratio of 6.0 to 53.5 to 40.5 (DPPA:DPPC:MPEG5000-DPPE) or a mole % ratio of 10 to 82 to 8 (10:82:8) (DPPA:DPPC:MPEG5000-DPPE).

In some embodiments where DPPC and DPPE and optionally MPEG5000-DPPE are used, their molar percentages may be about 77-90 mole % DPPC, about 5-15 mole % DPPE, and about 5-15 mole % MPEG5000-DPPE. Preferred ratios of each lipid include those described in the Examples section such as a weight % ratio of 6.0 to 53.5 to 40.5 (DPPE:DPPC:MPEG5000-DPPE) or a mole % ratio of 10 to 82 to 8 (10:82:8) (DPPE:DPPC:MPEG5000-DPPE).

### Gas

The gas is preferably substantially insoluble in the lipid solutions provided herein. The gas may be a non-soluble fluorinated gas such as sulfur hexafluoride or a perfluorocarbon gas. Examples of perfluorocarbon gases include perfluoropropane, perfluoromethane, perfluoroethane, perfluorobutane, perfluoropentane, perfluorohexane. Examples of gases that may be used in the microspheres of the invention are described in U.S. Pat. No. 5,656,211 and are incorporated by reference herein. In an important embodiment, the gas is perfluoropropane.

Examples of gases include, but are not limited to, hexafluoroacetone, isopropylacetylene, allene, tetrafluoroallene, boron trifluoride, 1,2-butadiene, 1,3-butadiene, 1,2,3-trichlorobutadiene, 2-fluoro-1,3-butadiene, 2-methyl-1,3 butadiene, hexafluoro-1,3-butadiene, butadiyne, 1-fluorobutane, 2-methylbutane, decafluorobutane (perfluorobutane), decafluoroisobutane (perfluoroisobutane), 1-butene, 2-butene, 2-methy-1-butene, 3-methyl-1-butene, perfluoro-1-butene, perfluoro-1-butene, perfluoro-2-butene, 4-phenyl-3-butene-2-one, 2-methyl-1-butene-3-yne, butylnitrate, 1-butyne, 2-butyne, 2-chloro-1,1,1,4,4,4-hexafluoro-butyne, 3-methyl-1-butyne, perfluoro-2-butyne, 2-bromo-butyraldehyde, carbonyl sulfide, crotononitrile, cyclobutane, methylcyclobutane, octafluorocyclobutane (perfluorocyclobutane), perfluoroisobutane, 3-chlorocyclopentene, cyclopropane, 1,2-dimethylcyclopropane, 1,1-dimethylcyclopropane, ethyl cyclopropane, methylcyclopropane, diacetylene, 3-ethyl-3-methyldiaziridine, 1,1,1-trifluorodiazoethane, dimethylamine, hexafluorodimethylamine, dimethylethylamine, bis-(dimethyl phosphine)amine, 2,3-dimethyl-2-norbornane, perfluoro-dimethylamine, dimethyloxonium chloride, 1,3-dioxolane-2-one, 1,1,1,1,2-tetrafluoroethane, 1,1,1-trifluoroethane, 1,1,2,2-tetrafluoroethane, 1,1,2-trichloro-1,2,2-trifluoroethane, 1,1-dichloroethane, 1,1-dichloro-1,2,2,2-tetrafluoroethane, 1,2-difluoroethane, 1-chloro-1,1,2,2,2-pentafluoroethane, 2-chloro-1,1-difluoroethane, 1-chloro-1,1,2,2-tetrafluoro-ethane, 2-chloro-1,1-difluoroethane, chloroethane, chloropentafluoroethane, dichlorotrifluoroethane, fluoroethane, nitropentafluoroethane, nitrosopentafluoro-ethane, perfluoroethane, perfluoroethylamine, ethyl vinyl ether, 1,1-dichloroethylene, 1,1-dichloro-1,2-difluoro-ethylene, 1,2-difluoroethylene, methane, methane-sulfonyl-chlori-detrifluoro, methane-sulfonyl-fluoride-trifluoro, methane-(pentafluorothio)trifluoro, methane-bromo-difluoro-nitroso, methane-bromo-fluoro, methane-bromo-chloro-fluoro, methane-bromo-trifluoro, methane-chloro-difluoro-nitro, methane-chloro-dinitro, methane-chloro-fluoro, methane-chloro-trifluoro, methane-chloro-difluoro, methane-dibromo-difluoro, methane-dichloro-difluoro, methane-dichloro-fluoro, methane-difluoro, methane-difluoro-iodo, methane-disilano, methane-fluoro, methane-iodomethane-iodo-trifluoro, methane-nitro-trifluoro, methane-nitroso-triofluoro, methane-tetrafluoro, methane-trichloro-fluoro, methane-trifluoro, methanesulfenylchloride-trifluoro, 2-methyl butane, methyl ether, methyl isopropyl ether, methyl lactate, methyl nitrite, methyl sulfide, methyl vinyl ether, neopentane, nitrogen (N.sub.2), nitrous oxide, 1,2,3-nonadecane tricarboxylic acid-2-hydroxycrimethylester, 1-nonene-3-yne, oxygen (O.sub.2), oxygen 17 (.sup.17 O.sub.2), 1,4-pentadiene, n-pentane, dodecafluoropentane (perfluoropentane), tetradecafluorohexane (perfluorohexane), perfluoroisopentane, perfluoroneopentane, 2-pentanone-4-amino-4-methyl, 1-pentene, 2-pentene {cis}, 2-pentene {trans}, 1-pentene-3-bromo, 1-pentene-perfluoro, phthalic acid-tetrachloro, piperidine-2,3,6-trimethyl, propane, propane-1,1,1,2,2,3-hexafluoro, propane-1,2-epoxy, propane-2,2 difluoro, propane-2-amino, propane-2-chloro, propane-heptafluoro-1-nitro, propane-heptafluoro-1-nitroso, perfluoropropane, propene, propyl-1,1,1,2,3,3-hexafluoro-2,3 dichloro, propylene-1-chloro, propylene-chloro-{trans}, propylene-2-chloro, propylene-3-fluoro, propylene-perfluoro, propyne, propyne-3,3,3-trifluoro, styrene-3-fluoro, sulfur hexafluoride, sulfur (di)-decafluoro(S.sub.2 F.sub.10), toluene-2,4-diamino, trifluoroacetonitrile, trifluoromethyl peroxide, trifluoromethyl sulfide, tungsten hexafluoride, vinyl acetylene, vinyl ether, neon, helium, krypton, xenon (especially rubidium enriched hyperpolarized xenon gas), carbon dioxide, helium, and air.

Fluorinated gases (that is, a gas containing one or more fluorine molecules, such as sulfur hexafluoride), fluorocarbon gases (that is, a fluorinated gas which is a fluorinated carbon or gas), and perfluorocarbon gases (that is, a fluorocarbon gas which is fully fluorinated, such as perfluoropropane and perfluorobutane) are preferred.

The gas such as the perfluorocarbon gas is typically present below its pure concentration at room temperature due to the incorporation of air during production. The concentration of perfluoropropane when present in a vial comprising a non-aqueous mixture and a gas headspace is expected to be about 6.52 mg/mL, at about one atmosphere of pressure. The concentrations of other gases, as known in the art, would be similarly diluted due to incorporation of air during production.

The gas, such as perflutren gas, may be injected into or otherwise added to the container (e.g., the vial) comprising the solution or into the solution itself in order to provide a gas other than air. Gases that are not heavier than air may be added to a sealed container while gases heavier than air may be added to a sealed or an unsealed container.

It will be understood by one skilled in the art that a gaseous precursor may also be used, followed by conversion of the precursor into a gas either by temperature or pressure change.

This disclosure therefore provides a number of inventive embodiments, as provided below in clause form:
Clause 1. An apparatus for forming gas-filled microspheres, the apparatus comprising:
   a holder arranged to hold a container comprising an ultrasound contrast agent (UCA) formulation; and
   a shaking means arranged to shake the holder at at least one speed, wherein the shaking means includes a motor arranged to drive movement of the holder via a transmission, wherein at least a portion of the transmission is arranged to prevent slippage during an activation of the UCA formulation, optionally wherein the transmission is arranged to create friction and/or resistance to movement.
Clause 2. The apparatus of clause 1, wherein the transmission includes at least one of a toothed, perforated, indented belt or track, a drive chain, an O-ring, or a gear box.
Clause 3. The apparatus of clause 1, wherein the transmission includes a toothed belt operably connected to each of the holder and the motor.
Clause 4. The apparatus of clause 3, wherein the transmission includes a gear having first and second tooth wheels, the first toothed wheel being attachable to the holder and the second toothed wheel being attachable to the motor.
Clause 5. The apparatus of clause 4, wherein the toothed belt is arranged to engage with each of the first and second toothed wheels.
Clause 6. The apparatus of clause 5, wherein the first and second toothed wheels are substantially parallel to one another.
Clause 7. The apparatus of clause 6, wherein the first and second toothed wheels are substantially co-planar with one another.
Clause 8. The apparatus of clause 4, wherein the transmission includes first and second O-rings, wherein the first O-ring is positioned on a first lateral side of the first toothed wheel and the second O-ring is positioned on a second lateral side of the first toothed wheel.
Clause 9. The apparatus of clause 8, wherein each of the first and second O-rings and the first toothed wheel are positioned around a spindle attachable to the holder.
Clause 10. The apparatus of clause 2, wherein the transmission includes a drive chain operably connected to each of the holder and the motor.
Clause 11. The apparatus of clause 10, wherein the transmission includes first and second profiled wheels, the first profiled wheel being attachable to the holder and the second profiled wheel being attachable to the motor.
Clause 12. The apparatus of clause 11, wherein the first and second profiled wheels include first and second sprockets, wherein the drive chain is arranged to engage with each of the first and second sprockets.
Clause 13. The apparatus of clause 2, wherein the transmission includes two or more 0-rings and the shaking means includes first and pulleys attachable to the holder and motor, respectively, the two or more O-rings arranged to engage with each of the first and second pulleys.
Clause 14. The apparatus of any one of clauses 1-13, further comprising a housing, the shaking means being disposed in the housing.
Clause 15. The apparatus of any one of clauses 1-14, further comprising a first identification means arranged to identify the UCA formulation in the container.
Clause 16. The apparatus of clause 15, wherein the first identification means includes at least one of an RFID reader, a microchip reader, and a barcode scanner.
Clause 17. The apparatus of clause 15, wherein the first identification means includes an antenna.
Clause 18. The apparatus of any one of clauses 1-17, wherein the holder includes a shaker arm.
Clause 19. The apparatus of any one of clauses 1-18, wherein the at least one speed is pre-determined.
Clause 20. The apparatus of any one of clauses 1-18, wherein the at least one speed is pre-set by the apparatus.
Clause 21. The apparatus of any one of clauses 1-20, wherein the at least one speed is less than 4830 rpm.
Clause 22. The apparatus of any one of clauses 1-20, wherein the at least one speed is between about 4830 rpm and about 6000 rpm.
Clause 23. The apparatus of any one of clauses 1-21, wherein the at least one speed is about 4530 rpm.
Clause 24. The apparatus of any one of clauses 1-20 and 22, wherein the at least one speed is about 4950 rpm.
Clause 25. The apparatus of any one of clauses 1-18, wherein the at least one speed includes a first speed of about 4530 rpm and a second speed of about 4950 rpm.
Clause 26. The apparatus of any one of clauses 1-18, wherein the apparatus is arranged to shake the holder at two or more speeds, wherein the two or more speeds are between about 4300 rpm and about 6000 rpm, and optionally between about 4300 rpm and about 5000 rpm.
Clause 27. The apparatus of clause 15, wherein the first identification means is further arranged to identify the at least one shaking speed of the UCA formulation in the container.
Clause 28. The apparatus of clause 17, wherein the identification means is arranged to identify the UCA formulation and/or the at least one shaking speed for the UCA formulation by reading an indicator on the container.
Clause 29. The apparatus of any one of clauses 1-28, wherein the motor includes a direct current (DC) motor, optionally a brushless DC motor.
Clause 30. A shaking device for forming gas-filled microspheres, the shaking device comprising:
   a holder arranged to hold a container comprising an ultrasound contrast agent (UCA) formulation;
   an identification means arranged to identify the UCA formulation in the container; and
   a shaking means arranged to shake the holder at at least one speed, wherein the shaking means includes a motor arranged to drive movement of the holder via a transmission, wherein the transmission includes at least one of a toothed, perforated, indented belt or track, a drive chain, and a gear box.
Clause 31. The shaking device of clause 30, wherein the transmission includes a gear having first and second toothed wheels, the first toothed wheel being attachable to the holder and the second toothed wheel being attachable to the motor.
Clause 32. The shaking device of clause 31, wherein the toothed belt is arranged to engage with each of the first and second toothed wheels.
Clause 33. The shaking device of clause 30, wherein the transmission includes first and second sprockets, the first sprocket being attachable to the holder and the second sprocket being attachable to the motor.
Clause 34. The shaking device of clause 33, wherein the drive chain is arranged to engage with each of the first and second sprockets.
Clause 35. The shaking device of any one of clauses 30-34, further comprising a housing, the shaking means being disposed in the housing.
Clause 36. The shaking device of any one of clauses 30-35, further comprising a first identification means arranged to identify the UCA formulation in the container.
Clause 37. The shaking device of clause 36, wherein the first identification means includes at least one of an RFID reader, a microchip reader, and a barcode scanner.
Clause 38. The shaking device of clause 36, wherein the first identification means includes at least one antenna.
Clause 39. The shaking device of clause 36, wherein the first identification means is further arranged to identify a shaking speed for the UCA formulation in the container.
Clause 40. The shaking device of clause 36, wherein the first identification means identifies the UCA formulation and/or the shaking speed for the UCA formulation by reading an indicator on the container.
Clause 41. The shaking device of any one of clauses 30-41, wherein the holder includes a shaker arm.
Clause 42. The shaking device of any one of clauses 30-41, wherein the at least one speed speeds is pre-determined.
Clause 43. The shaking device of any one of clauses 30-41, wherein the at least one speed is pre-set by the shaking device.
Clause 44. The shaking device of any one of clauses 30-43, wherein the at least one speed is less than 4830 rpm.
Clause 45. The shaking device of any one of clauses 30-43, wherein the at least one speed is between about 4830 rpm and about 6000 rpm.
Clause 46. The shaking device of any one of clauses 30-44, wherein the at least one speed is about 4530 rpm.
Clause 47. The shaking device of any one of clauses 30-43 and 46, wherein the at least one speed is about 4950 rpm.
Clause 48. The shaking device of any one of clauses 30-43, wherein the at least one speed includes a first speed of about 4530 rpm and a second speed of about 4950 rpm.
Clause 49. The shaking device of any one of clauses 30-43, wherein the shaking device is arranged to shake the holder at two or more speeds, wherein the two or more speeds are between about 4300 rpm and about 6000 rpm and optionally between about 4300 rpm and about 5000 rpm.
Clause 50. The shaking device of any one of clauses 30-49, wherein the motor is a direct current (DC) motor, optionally a brushless DC motor.
Clause 51. A method of using gas-filled lipid microspheres for ultrasound contrast imaging comprising
   activating an ultrasound contrast agent (UCA) formulation to form gas-filled lipid microspheres using a shaking device having a shaking means including a motor arranged to drive movement of a holder via a transmission, wherein the holder is arranged to shake a container with the UCA formulation at at least one shaking speed, wherein at least a portion of the transmission is arranged to prevent slippage during activation of the UCA formulation, optionally wherein the transmission is arranged to create friction and/or resistance to movement;
   administering the gas-filled lipid microspheres to a subject, and
   obtaining an ultrasound contrast image of the subject.
Clause 52. The method of clause 51, wherein the container comprises an indicator on its external surface.
Clause 53. The method of clause 51, further comprising identifying the UCA formulation prior to the step of activating.
Clause 54. The method of clause 53, wherein identifying includes reading the indicator on the external surface of the vial via an identification means.
Clause 55. The method of clause 54, wherein the identification means includes at least one of an RFID reader, a microchip reader, and a barcode scanner.
Clause 56. The method of clause 54, wherein the identification means includes at least one antenna.
Clause 57. The method of clause 53, further comprising identifying the at least one shaking speed for the UCA formulation in the container, optionally using the shaking device.
Clause 58. The method of clause 51, wherein the at least one shaking speed is pre-determined.
Clause 59. The method of clause 51, wherein the at least one shaking speed is pre-set by the shaking device.
Clause 60. The method of clause 51, wherein the at least one shaking speed is less than 4830 rpm.
Clause 61. The method of clause 51, wherein the at least one shaking speed is between about 4830 rpm and about 6000 rpm.
Clause 62. The method of clause 51, wherein the at least one shaking speed is about 4530 rpm.
Clause 63. The method of clause 51, wherein the at least one shaking speed is about 4950 rpm.
Clause 64. The method of clause 51, wherein the at least one speed includes a first speed of about 4530 rpm and a second speed of about 4950 rpm.
Clause 65. The method of clause 53, further comprising identifying two or more shaking speeds for the UCA formulation in the container, optionally using the shaking device, wherein the two or more speeds are between about 4300 rpm and about 6000 rpm or optionally between about 4300 rpm and about 5000 rpm.
Clause 66. The method of clause 51, wherein activating includes shaking the container via the holder.
Clause 67. The method of clause 51, wherein the holder includes a shaker arm.
Clause 68. A method of using gas-filled lipid microspheres to ultrasound contrast image a subject, the method comprising:
   identifying an ultrasound contrast agent (UCA) formulation;
   activating the UCA formulation to form gas-filled lipid microspheres using a shaking device having a holder arranged to hold a container comprising the UCA formulation, and a shaking means arranged to drive movement of the holder via a motor and a transmission, the transmission including at least one of a tooth belt, a drive chain, and a gearbox;
   administering the gas-filled lipid microspheres into a subject; and
   obtaining an ultrasound contrast image of the subject.
Clause 69. The method of clause 68, wherein the container includes an indicator on its external surface.
Clause 70. The method of clause 68, wherein identifying includes reading the indicator on the external surface of the container via an identification means that is optionally comprised in shaking device.
Clause 71. The method of clause 70, wherein the identification means includes at least one of an RFID reader, a microchip reader, and a barcode scanner.
Clause 72. The method of clause 70, wherein the identification means includes at least one antenna.
Clause 73. The method of clause 68, further comprising identifying a shaking speed for the UCA formulation in the container via the identification means, optionally using the identification means.
Clause 74. The method of clause 68, wherein activating includes shaking the container via the holder.
Clause 75. The method of clause 74, wherein the holder includes a shaker arm.
Clause 76. A method of using gas-filled lipid microspheres for ultrasound contrast imaging comprising
   activating an ultrasound contrast agent (UCA) formulation to form gas-filled lipid microspheres using the apparatus of any one of clauses 1-29;
   administering the gas-filled lipid microspheres to a subject, and
   obtaining an ultrasound contrast image of the subject.
Clause 77. A method of using gas-filled lipid microspheres for ultrasound contrast imaging comprising
   activating an ultrasound contrast agent (UCA) formulation to form gas-filled lipid microspheres using the shaking device of any one of clauses 30-50;
   administering the gas-filled lipid microspheres to a subject, and
   obtaining an ultrasound contrast image of the subject.
Clause 78. The method of any one of clauses 51-77, wherein the UCA formulation comprises DPPA, DPPC and MPEG5000-DPPE.
Clause 79. The method of any one of clauses 51-78, wherein the ultrasound contrast agent (UCA) formulation is in a the vial and the vial further comprises perfluoropropane gas, and the gas-filled microspheres are perfluoropropane gas filled microspheres.
Clause 80. The apparatus of clause 17, wherein the at least one antenna includes first and second antennas, optionally wherein the first and second antennas are arranged to read an indicator on the container no matter where along a figure of eight path the holder is positioned when a second container is inserted into the holder.
Clause 81. The shaking device of clause 38, wherein the at least one antenna includes first and second antennas, optionally wherein the first and second antennas are arranged to read an indicator on the container no matter where along a figure of eight path the holder is positioned when a second container is inserted into the holder.
Clause 82. The method of clause 56 or 70, wherein the at least one antenna is arranged to read an indicator on the container no matter where along a figure of eight path the holder is positioned when a second container is inserted into the holder.
Clause 83. The apparatus of clause 1 or 30, wherein the transmission is arranged to reduce resonance such that the motor does not labor at certain speeds and is able to maintain consistent activation of the UCA formulation.
Clause 84. The method of clause 51 or 68, wherein the transmission is arranged to reduce resonance such that the motor does not labor at certain speeds and is able to maintain consistent activation of the UCA formulation.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An apparatus for forming gas-filled microspheres, the apparatus comprising:
a holder arranged to hold a container comprising an ultrasound contrast agent (UCA) formulation; and
a shaking means arranged to shake the holder at at least one speed, wherein the shaking means includes a motor arranged to drive movement of the holder via a transmission, wherein at least a portion of the transmission is arranged to prevent slippage during an activation of the UCA formulation, optionally wherein the transmission is arranged to create friction and/or resistance to movement.

2. The apparatus of claim 1, wherein the transmission includes at least one of a toothed, perforated, indented belt or track, a drive chain, an O-ring, or a gear box.

3. The apparatus of claim 1, wherein the transmission includes a toothed belt operably connected to each of the holder and the motor.

4. The apparatus of claim 3, wherein the transmission includes a gear having first and second tooth wheels, the first toothed wheel being attachable to the holder and the second toothed wheel being attachable to the motor.

5. The apparatus of claim 4, wherein the toothed belt is arranged to engage with each of the first and second toothed wheels.

6. The apparatus of claim 5, wherein the first and second toothed wheels are substantially parallel to one another.

7. The apparatus of claim 6, wherein the first and second toothed wheels are substantially co-planar with one another.

8. The apparatus of claim 4, wherein the transmission includes first and second O-rings, wherein the first O-ring is positioned on a first lateral side of the first toothed wheel and the second O-ring is positioned on a second lateral side of the first toothed wheel.

9. The apparatus of claim 8, wherein each of the first and second O-rings and the first toothed wheel are positioned around a spindle attachable to the holder.

10. The apparatus of claim 2, wherein the transmission includes a drive chain operably connected to each of the holder and the motor.

11. The apparatus of claim 10, wherein the transmission includes first and second profiled wheels, the first profiled wheel being attachable to the holder and the second profiled wheel being attachable to the motor.

12. The apparatus of claim 11, wherein the first and second profiled wheels include first and second sprockets, wherein the drive chain is arranged to engage with each of the first and second sprockets.

13. The apparatus of claim 2, wherein the transmission includes two or more O-rings and the shaking means includes first and pulleys attachable to the holder and motor, respectively, the two or more O-rings arranged to engage with each of the first and second pulleys.

14. The apparatus of any one of claims 1-13, further comprising a first identification means arranged to identify the UCA formulation in the container.

15. The apparatus of claim 14, wherein the first identification means includes at least one of an RFID reader, a microchip reader, and a barcode scanner.

16. The apparatus of claim 14, wherein the first identification means includes an antenna.

17. The apparatus of any one of claims 1-16, wherein the apparatus is arranged to shake the holder at two or more speeds, wherein the two or more speeds are between about 4300 rpm and about 6000 rpm, and optionally between about 4300 rpm and about 5000 rpm.

18. The apparatus of any one of claims 1-17, wherein the motor includes a direct current (DC) motor, optionally a brushless DC motor.

19. A shaking device for forming gas-filled microspheres, the shaking device comprising:
a holder arranged to hold a container comprising an ultrasound contrast agent (UCA) formulation;
an identification means arranged to identify the UCA formulation in the container; and
a shaking means arranged to shake the holder at at least one speed, wherein the shaking means includes a motor arranged to drive movement of the holder via a transmission, wherein the transmission includes at least one of a toothed, perforated, indented belt or track, a drive chain, and a gear box.

20. The shaking device of claim 19, wherein the transmission includes a gear having first and second toothed wheels, the first toothed wheel being attachable to the holder and the second toothed wheel being attachable to the motor.

21. The shaking device of claim 20, wherein the toothed belt is arranged to engage with each of the first and second toothed wheels.

22. The shaking device of claim 19, wherein the transmission includes first and second sprockets, the first sprocket being attachable to the holder and the second sprocket being attachable to the motor.

23. The shaking device of claim 22, wherein the drive chain is arranged to engage with each of the first and second sprockets.

24. The shaking device of claim 19, wherein the identification means includes at least one of an RFID reader, a microchip reader, and a barcode scanner.

25. The shaking device of claim 19, wherein the identification means includes at least one antenna.

26. The shaking device of any one of claims 19-25, wherein the shaking device is arranged to shake the holder at two or more speeds, wherein the two or more speeds are between about 4300 rpm and about 6000 rpm and optionally between about 4300 rpm and about 5000 rpm.

27. The shaking device of any one of claims 19-26, wherein the motor is a direct current (DC) motor, optionally a brushless DC motor.

28. A method of using gas-filled lipid microspheres for ultrasound contrast imaging comprising
activating an ultrasound contrast agent (UCA) formulation to form gas-filled lipid microspheres using a shaking device having a shaking means including a motor arranged to drive movement of a holder via a transmission, wherein the holder is arranged to shake a container with the UCA formulation at at least one shaking speed, wherein at least a portion of the transmission is arranged to prevent slippage during activation of the UCA formulation, optionally wherein the transmission is arranged to create friction and/or resistance to movement;
administering the gas-filled lipid microspheres to a subject, and
obtaining an ultrasound contrast image of the subject.

29. A method of using gas-filled lipid microspheres to ultrasound contrast image a subject, the method comprising:
identifying an ultrasound contrast agent (UCA) formulation;
activating the UCA formulation to form gas-filled lipid microspheres using a shaking device having a holder arranged to hold a container comprising the UCA formulation, and a shaking means arranged to drive movement of the holder via a motor and a transmission, the transmission including at least one of a tooth belt, a drive chain, and a gearbox;
administering the gas-filled lipid microspheres into a subject; and
obtaining an ultrasound contrast image of the subject.

30. A method of using gas-filled lipid microspheres for ultrasound contrast imaging comprising
activating an ultrasound contrast agent (UCA) formulation to form gas-filled lipid microspheres using the apparatus of any one of claims 1-18;
administering the gas-filled lipid microspheres to a subject, and
obtaining an ultrasound contrast image of the subject.

31. A method of using gas-filled lipid microspheres for ultrasound contrast imaging comprising
activating an ultrasound contrast agent (UCA) formulation to form gas-filled lipid microspheres using the shaking device of any one of claims 19-27;
administering the gas-filled lipid microspheres to a subject, and
obtaining an ultrasound contrast image of the subject.

32. The apparatus of claim 16, wherein the at least one antenna includes first and second antennas, optionally wherein the first and second antennas are arranged to read an indicator on the container no matter where along a figure of eight path the holder is positioned when a second container is inserted into the holder.

33. The shaking device of claim 25, wherein the at least one antenna includes first and second antennas, optionally wherein the first and second antennas are arranged to read an indicator on the container no matter where along a figure of eight path the holder is positioned when a second container is inserted into the holder.

34. The apparatus of claim 1 or the shaking device of claim 19, wherein the transmission is arranged to reduce resonance such that the motor does not labor at certain speeds and is able to maintain consistent activation of the UCA formulation.

35. The method of claim 28 or 29, wherein the transmission is arranged to reduce resonance such that the motor does not labor at certain speeds and is able to maintain consistent activation of the UCA formulation.
